SEQUENCE LISTING (48 sequences)

SEQ ID NO: 1 - DNA, 1155 bp, Pseudomonas resinovorans
gtggcgaacgttgatgaggcaattttaaaaagagtaaaaggctgggcgccctacgtggatgcgaagctaggctttcgcaatcattggtacccggtgatgttttcgaaagagatcgacgagggcgagccgaagacactaaaactgctcggtgagaacttgctcgtcaatcgtatcgatgggaagctgtattgcctcaaggaccgctgcctgcatcgcggcgtccagttgtcggtcaaagtcgagtgcaaaacgaagtcgacgatcacatgctggtaccacgcgtggacctatcgctgggaagacggcgttctgtgcgacatcttgacgaatccgacaagcgcacagatcggtcgacaaaagctgaaaacttacccagtgcaggaagccaagggctgcgtcttcatttatcttggcgatggcgaccctcctcccttggcccgcgatacgccacccaatttccttgacgatgacatggaaatcctcgggaagaaccaaatcatcaagtctaactggcgcctcgctgtggaaaacggtttcgatccgagccacatttatattcacaaagactcaattctggtcaaggacaacgatcttgccttgccactaggtttcgcgccaggaggggatcgaaagcaacaaactcgtgtggttgacgatgacgtcgtcggacgcaagggtgtttacgatcttattggcgaacatggggtcccagtgtttgagggaactatcgggggcgaagtggtccgcgaaggtgcctacggcgaaaaaattgtagcgaacgatatctccatttggctcccgggtgttctcaaggtcaatccgttccccaatccggacatgatgcagttcgagtggtacgtgccgattgacgaaaacacacactattacttccaaactcttggcaaaccatgtgccaatgacgaggaacggaagaattacgaacaagagttcgaaagcaagtggaaaccgatggcgctcgaaggattcaacaacgatgacatctgggctcgcgaagctatggtggatttctacgccgatgataaaggctgggtcaacgagattttgttcgaggtggacgaggctatcgtggcatggcgcaagctggcgagcgaacacaatcagggtattcagacccaagcgcacgtttcgggctga

SEQ ID NO: 2 - protein, 384 aa, Pseudomonas resinovorans
MANVDEAILKRVKGWAPYVDAKLGFRNHWYPVMFSKEIDEGEPKTLKLLGENLLVNRIDGKLYCLKDRCLHRGVQLSVKVECKTKSTITCWYHAWTYRWEDGVLCDILTNPTSAQIGRQKLKTYPVQEAKGCVFIYLGDGDPPPLARDTPPNFLDDDMEILGKNQIIKSNWRLAVENGFDPSHIYIHKDSILVKDNDLALPLGFAPGGDRKQQTRVVDDDVVGRKGVYDLIGEHGVPVFEGTIGGEVVREGAYGEKIVANDISIWLPGVLKVNPFPNPDMMQFEWYVPIDENTHYYFQTLGKPCANDEERKNYEQEFESKWKPMALEGFNNDDIWAREAMVDFYADDKGWVNEILFEVDEAIVAWRKLASEHNQGIQTQAHVSG

SEQ ID NO: 3 - DNA, 324 bp, Pseudomonas resinovorans
atgaaccaaatttggttgaaagtatgtgctgcatctgacatgcaacctggcacgatacgtcgcgtcaaccgcgtaggtgctgcacctctcgcagtctatcgtgttggcgatcagttctacgccactgaagatacgtgcacgcatggtattgcttcgctttcggaagggacactcgatggtgacgtgattgaatgtccctttcacggcggcgccttcaatgtttgtaccggcatgccggcatcaagtccatgtacagtgccgctaggagtgttcgaggtagaagtcaaagagggcgaagtttatgtcgccggagaaaagaagtag

SEQ ID NO: 4 - protein, 107 aa, Pseudomonas resinovorans
MNQIWLKVCAASDMQPGTIRRVNRVGAAPLAVYRVGDQFYATEDTCTHGIASLSEGTLDGDVIECPFHGGAFNVCTGMPASSPCTVPLGVFEVEVKEGEVYVAGEKK

SEQ ID NO: 5 - DNA, 990 bp, Pseudomonas resinovorans
atgtaccaactcaaaattgaagggcaagcgccagggacctgcggctcagggaagagcctgttggtctcagcacttgctaatggtatcggatttccgtacgagtgtgcatcgggaggttgcggagtatgcaaattcgagttactcgaagggaatgtccaatcaatgtggccggatgctccaggactttcttcgcgagatcgtgagaagggcaaccgccatcttgcatgccagtgcgttgcgctctcagacctgcggatcaaagtcgcagtgcaggacaagtacgtcccaacgattccaatctcaagaatggaagcggaagttgttgaggtccgggcgctaactcatgacctgctgtccgtgcgattacgcactgatgggccagcaaatttcctccccggccagttctgcctagtagaggcagagcagttgccaggcgtggttcgcgcatattcaatggcgaatttaaagaaccccgaaggcatatgggagttctatattaagagggtacccacaggacgatttagtccttggcttttcgaaaatagaaaagaaggcgctcgtctatttttgacgggaccaatgggcacatctttcttccgtccagggaccggccgaaagagtctttgcattggcggcggtgccgggctctcgtatgcggccgctattgcacgcgcctcgatgcgcgaaacagacaagccggtaaagttgttctacggctcaagaactccgcgcgacgctgttcggtggatcgatatcgacatcgatgaggacaagcttgaggtcgtccaggcagttacggaagacacggatagcctttggcaagggcccactggttttattcatcaggttgtcgacgcagcgctgcttgaaaccctaccggaatacgaaatttatcttgccggtccaccgcctatggtcgacgctactgtccgtatgctgctcggcaagggtgttccacgcgatcaaattcattttgacgcatttttctaa

SEQ ID NO: 6 - protein, 329 aa, Pseudomonas resinovorans
MYQLKIEGQAPGTCGSGKSLLVSALANGIGFPYECASGGCGVCKFELLEGNVQSMWPDAPGLSSRDREKGNRHLACQCVALSDLRIKVAVQDKYVPTIPISRMEAEVVEVRALTHDLLSVRLRTDGPANFLPGQFCLVEAEQLPGVVRAYSMANLKNPEGIWEFYIKRVPTGRFSPWLFENRKEGARLFLTGPMGTSFFRPGTGRKSLCIGGGAGLSYAAAIARASMRETDKPVKLFYGSRTPRDAVRWIDIDIDEDKLEVVQAVTEDTDSLWQGPTGFIHQVVDAALLETLPEYEIYLAGPPPMVDATVRMLLGKGVPRDQIHFDAFF

SEQ ID NO: 7 - DNA, 1137 bp, Sphingomonas sp. KA1
gtggctaaccaaccatcaatcgccgagcgcagaaccaaggtttgggagccttatatccgtgcgaaactcgggttccgaaaccattggtatcccgttcgcctcgcgagcgaaatcgccgaaggtactcccgttcccgtcaagctcctgggagagaagattctgctcaatcgcgtgggcggcaaggtctatgcgatccaggacaggtgcctgcatcgcggtgtaacgctttccgaccgggtcgagtgctattccaagaacaccatatcctgctggtatcacggctggacatatcgctgggacgatggccgcctcgtcgatatcctcacaaaccccggcagtgtgcagatcggccggcgcgctttgaagacgttcccggttgaagaggccaaaggtcttatcttcgtttacgtaggcgacggcgaaccaacgccgcttatcgaagatgtgccgcccggcttccttgatgaaaaccgcgccattcacggccaacatcggctcgtggcctcgaactggcgcttgggtgcggaaaacggctttgatgcggggcacgtcttcattcacaagaattcgatcctggtgaagggcaacgatatcattctgccgcttggctttgcgcctggcgatcccgaccagcttacgcgttccgaggttgctgcgggcaagcccaaaggtgtttacgatctgcttggcgagcattcggtgccggttttcgaaggcatgatcgaaggcaaacctgcaatccatggcaacattggcagcaagcgcgtcgccatcagcatatcgatctggctgccgggcgtactcaaggtcgaaccgtggccggatcccgagctcacgcagttcgaatggtacgtgccggtcgatgagaccagccacctctacttccagacgctgggcaaagtcgtgacgtcaaaggaagcggcagactccttcgagcgagaattccacgaaaaatgggtaggcctcgcgcttaacggcttcaatgatgacgacatcatggcacgtgaatcgatggagccgttctacgctgatgatcgcggttggtccgaagaaatcctgttcgagccggaccgcgcaatcatcgagtggcgggggcttgccagtcagcacaatcgcggcattcaggaagcacgttga

SEQ ID NO: 8 - protein, 378 aa, Sphingomonas sp. KA1
MANQPSIAERRTKVWEPYIRAKLGFRNHWYPVRLASEIAEGTPVPVKLLGEKILLNRVGGKVYAIQDRCLHRGVTLSDRVECYSKNTISCWYHGWTYRWDDGRLVDILTNPGSVQIGRRALKTFPVEEAKGLIFVYVGDGEPTPLIEDVPPGFLDENRAIHGQHRLVASNWRLGAENGFDAGHVFIHKNSILVKGNDIILPLGFAPGDPDQLTRSEVAAGKPKGVYDLLGEHSVPVFEGMIEGKPAIHGNIGSKRVAISISIWLPGVLKVEPWPDPELTQFEWYVPVDETSHLYFQTLGKVVTSKEAADSFEREFHEKWVGLALNGFNDDDIMARESMEPFYADDRGWSEEILFEPDRAIIEWRGLASQHNRGIQEAR

SEQ ID NO: 9 - DNA, 330 bp, Sphingomonas sp. KA1
atgaccgcaaaggtccgcgtgatcttccgcgcagccggcggcttcgagcatctggtcgaaaccgaagcgggagtatcgctcatggaagcggccgttctgaacggcgtggacggtatcgaagccgtttgcgggggcgcctgtgcctgcgccacgtgccacgtttacgttggccccgagtggctagatgcgctgaaaccgccgagtgagaccgaagacgaaatgctcgattgcgtagcggaacgtgcgccgcattcgcggctgtcctgccagatccgccttaccgacctgctcgacggcctgaccctggaactgccgaaggcacagtcatga

SEQ ID NO: 10 - protein, 109 aa, Sphingomonas sp. KA1
MTAKVRVIFRAAGGFEHLVETEAGVSLMEAAVLNGVDGIEAVCGGACACATCHVYVGPEWLDALKPPSETEDEMLDCVAERAPHSRLSCQIRLTDLLDGLTLELPKAQS

SEQ ID NO: 11 - DNA, 1224 bp, Sphingomonas sp. KA1
atgatcacatatgatgttgtcatcgtgggcgccggccacggtggcgcccaggcggcgatagcgttacgccagcgtcacttcgagggatcgatcgcggtgatcggcgaggagcctgatctgccctatgagcggccgcctctcagtaaggactatctctcggggaagaaagcgttcgagcgcatactcatccgcccggccaccttttgggaggaacgcggtgtgaggatgttgaccggcagacgcgtcgccgcggtcaatcctgccgcacataccgtctcgaccgacgatggagagagttttggttacggccgactgatctgggcagcgggtggacgcccccgccgcttgacatgcaccggccatgatctcgctggagtccatcaggtgcgcacccgcgccgatgtagaccagatgatcgtggagcttcctgaaacggctcgagtagcagtgatcggtggcggctatatcggcctggaagcggcagcggtccttgccgaaatggggaagcatgtgaccgtattggaggcgcaggaccgtgtcctcgcgcgtgtcgccggggaagccttgtcccgcttcttcgaagcggagcatcgggcgcacggggtcgacgtgcgattaggtgcagctgtcgattgcatcgagggacgcgacggccgggccgttggcgttcgcctcgccgatggaacgctggttgccgcggacatggtgattgtgggcatcggtatcgttccggcggtcgaacccttgttggctgcgggagcgcttggcatgaatggggtccaagtggacgagcatggccggacctcgttgcctgacattttcgcgatcggcgactgcgcgctgcatatcaatgcctttgccgacaatcttcctatccggcttgaatcggtccaaaacgccaacgatctcgcgacgaccgttgcccgaacactgaccggcgatccagaaccttacgtctcggtgccgtggttctggtccaaccaatatgatctgcgccttcagacggtaggactgtcggccggacatgacgcggcaataacgcgtggcaacccggtggaccgcagtttttccatcgtttatctcaaccagggccgggttatcgcgctcgattgcgtgaatgccgtcaaagactatgtccagggcaaggcgctggtcgcaactcgtgtcgcagcaagtcctgaggcgctatctgacccagcgctgccactgaaagcatttgtgtaa

SEQ ID NO: 12 - protein, 407 aa, Sphingomonas sp. KA1
MITYDVVIVGAGHGGAQAAIALRQRHFEGSIAVIGEEPDLPYERPPLSKDYLSGKKAFERILIRPATFWEERGVRMLTGRRVAAVNPAAHTVSTDDGESFGYGRLIWAAGGRPRRLTCTGHDLAGVHQVRTRADVDQMIVELPETARVAVIGGGYIGLEAAAVLAEMGKHVTVLEAQDRVLARVAGEALSRFFEAEHRAHGVDVRLGAAVDCIEGRDGRAVGVRLADGTLVAADMVIVGIGIVPAVEPLLAAGALGMNGVQVDEHGRTSLPDIFAIGDCALHINAFADNLPIRLESVQNANDLATTVARTLTGDPEPYVSVPWFWSNQYDLRLQTVGLSAGHDAAITRGNPVDRSFSIVYLNQGRVIALDCVNAVKDYVQGKALVATRVAASPEALSDPALPLKAFV

SEQ ID NO: 13 - DNA, 1137 bp, Sphingosinicella sp. JP1
gtggctaaccaaccatcaatcgccgagcgcagaaccaaggtttgggagccttacatccgtgcgaaactcgggttccggaaccattggtatcccgttcgcctcgtgagcgaaatcgccgaaggtgctcccgttcccgtcaagctcctgggagagaagattctgctcaatcgcgtgggaggcaaggtctatgcgatccaggacaggtgcctgcatcgcggtgtaacgctttccgaccgggtcgagtgctattccaggaacaccatatcctgctggtatcatggctggacatatcgctgggacgatggccgcctcgtcgatatcctcacaaacccgggcagtgtgcagatcggccggcgcgctttgaagacgttcccggttgaagaggccaaaggtcttatcttcgtttacgtaggcgacggcgagccaacgccgcttgtcgaagatgtaccgcccggtttccttgatgaaaaccgcgccattcacggccaacatcggctcgtggcctcgaactggcgcttgggtgcggaaaacggctttgatgcggggcacgtcttcatccacaagaattcgatcctggtgaagggcaacgatatcattctgccgcttggttttgcgcctggcgatcccgaccagcttacgcgttccgaggttgctgcgggcaagcccaagggtgtttacgatctgcttggcgagcattcggtgccggttttcgaaggcatgatcgaaggcgaacctgcaatccatggcaacattggcagcaagcgcgtcgcaatcagcatatcgatctggctgccgggcgtgctcaaggtcgaaccgtggccggatcccgagctcacgcagttcgaatggtacgtgccggtcgacgagaccagccacctctacttccagacgctgggcaaagtcgtgacgtcaaaggaagcggcagacttcttcgagcgagaattccacgaaaaatgggtaggcctcgcgcttaacggcttcaatgatgacgacatcatggcacgggaatcgatggagccgttctacgctgatgatcgcggttggtccgaagaaatcctgttcgagccggaccgcgcaatcatcgagtggcggcggcttgccagtcagcacaatcgcggcattcaggaagcacgttga

SEQ ID NO: 14 - protein, 376 aa, Sphingosinicella sp. JP1
VANQPSIAERRTKVWEPYIRAKLGFRNHWYPVRLVSEIAEGAPVPVKLLGEKILLNRVGGKVYAIQDRCLHRGVTLSDRVECYSRNTISCWYHGWTYRWDDGRLVDILTNPGSVQIGRRALKTFPVEEAKGLIFVYVGDGEPTPLVEDVPPGFLDENRAIHGQHRLVASNWRLGAENGFDAGHVFIHKNSILVKGNDIILPLGFAPGDPDQLTRSEVAAGKPKGVYDLLGEHSVPVFEGMIEGEPAIHGNIGSKRVAISISIWLPGVLKVEPWPDPELTQFEWYVPVDETSHLYFQTLGKVVTSKEAADFFEREFHEKWVGLALNGFNDDDIMARESMEPFYADDRGWSEEILFEPDRAIIEWRRLASQHNRGIQE

SEQ ID NO: 15 - DNA, 330 bp, Sphingosinicella sp. JP1
atgaccgcaaaggtccgcgtgatcttccgcgcagccggcggcttcgagcatctggtcgaaaccgaagcgggagtatcgctcatggaagcggccgttctgaacagcgtggacggtatcgaagccgtttgcgggggcgcctgcgcctgcgccacgtgccacgtttacgttgcccccgagtggctcgatgcgctgaaaccgccgagcgagaccgaagacgaaatgctcgattgcgtagcagaacgcgcgccgcattcgcggctgtcctgccagatccgccttaccgacctgctcgacggcctgaccctggaactgccgaaggcacagtcatga

SEQ ID NO: 16 - protein, 109 aa, Sphingosinicella sp. JP1
MTAKVRVIFRAAGGFEHLVETEAGVSLMEAAVLNSVDGIEAVCGGACACATCHVYVAPEWLDALKPPSETEDEMLDCVAERAPHSRLSCQIRLTDLLDGLTLELPKAQS

SEQ ID NO: 17 - DNA, 1224 bp, Sphingosinicella sp. JP1; identical to SEQ ID NO: 11

SEQ ID NO: 18 - protein, 407 aa, Sphingosinicella sp. JP1; identical to SEQ ID NO: 12

SEQ ID NO: 19 - DNA, 1167 bp, Nocardiodes aromaticivorans
atgagcacctctcaggaaatctccgaccctgcgcaggccacgagcagcgcgcaggtcaagtggccccgctacctcgaagcgacgctcggcttcgacaaccactggcatccggcagccttcgaccacgagctcgccgagggcgagttcgtcgcagtcacgatgctcggggagaaggtcctgctgactcgcgccaagggcgaggtcaaggccatcgccgacgggtgcgcccaccgtggcgtcccgttctccaaggagcctctgtgcttcaaggccggcaccgtctcctgctggtaccacggctggacctacgacctcgacgacggccgcctcgtcgacgtgctcacctctcccggttcgccggtcattggcaagatcggcatcaaggtctacccggtccaggtcgctcagggcgtcgtgttcgtcttcatcggcgacgaggagccccacgccctgagtgaggacctccccccgggcttcctcgacgaggacacccacttgctggggatccgtcggaccgtccagtcgaactggcgtctgggcgtggagaacggcttcgacaccactcacatcttcatgcaccgcaactccccgtgggtctcgggcaaccggctggcgttcccgtacggcttcgtccccgctgaccgtgacgcgatgcaggtttacgacgagaactggcctaagggtgttctcgaccggctctcggagaactacatgccggtcttcgaggcgaccctcgacggcgaaacggtccttagcgccgagctcaccggcgaagagaagaaggtcgccgcccaggtcagcgtgtggctgcccggcgtgctcaaggtcgacccgttcccggacccgaccctcatccagtacgagttctacgtgccgatctccgagacccagcacgagtacttccaggtgctccagcggaaggtcgagggacccgaggacgtcaagaccttcgaggtcgagttcgaggagcggtggcgcgacgacgccctgcacggcttcaatgacgacgacgtgtgggcgcgtgaggcccagcaagagttctacggcgaacgcgacggctggtccaaggagcagctgttcccgccggacatgtgcatcgtgaagtggcggaccctcgcctccgagcgcggccgcggcgtgcgtgcggcccgagtggaaatgtcgtga

SEQ ID NO: 20 - protein, 388 aa, Nocardiodes aromaticivorans
MSTSQEISDPAQATSSAQVKWPRYLEATLGFDNHWHPAAFDHELAEGEFVAVTMLGEKVLLTRAKGEVKAIADGCAHRGVPFSKEPLCFKAGTVSCWYHGWTYDLDDGRLVDVLTSPGSPVIGKIGIKVYPVQVAQGVVFVFIGDEEPHALSEDLPPGFLDEDTHLLGIRRTVQSNWRLGVENGFDTTHIFMHRNSPWVSGNRLAFPYGFVPADRDAMQVYDENWPKGVLDRLSENYMPVFEATLDGETVLSAELTGEEKKVAAQVSVWLPGVLKVDPFPDPTLIQYEFYVPISETQHEYFQVLQRKVEGPEDVKTFEVEFEERWRDDALHGFNDDDVWAREAQQEFYGERDGWSKEQLFPPDMCIVKWRTLASERGRGVRAARVEMS

SEQ ID NO: 21 - DNA, 348 bp, Nocardiodes aromaticivorans
atgaacaggcattcggcgggtcagtccaccccggtacgtgtcgccaccctcgaccagctcaagccgggggttcccacggccttcgacgtcgacggtgacgaggtgatggtggtgcgcgacggagacagcgtgtacgccatatccaacctctgcagtcatgccgaggcgtacttggacatgggtgtcttccacgccgaaagcctcgagatcgagtgcccgctccatgtcggccgcttcgatgtccggaccggcgcgccgaccgccttgccgtgcgtattgccggtccgtgcctacgacgtcgtcgtcgacgggaccgagatcctcgtggcgccgaaggaggcagactga

SEQ ID NO: 22 - protein, 115 aa, Nocardiodes aromaticivorans
MNRHSAGQSTPVRVATLDQLKPGVPTAFDVDGDEVMVVRDGDSVYAISNLCSHAEAYLDMGVFHAESLEIECPLHVGRFDVRTGAPTALPCVLPVRAYDVVVDGTEILVAPKEAD

SEQ ID NO: 23 - DNA, 1173 bp, Nocardiodes aromaticivorans
atgcgccgccattacgagtacctggtcgtcggtggtggcgtcgccggcggtcgcgcggtcgaagcgctgtcaaagcgcgccgactcggtcgccctcgtcagcgcggaacactggcgaccctatgcgcgaccgccactgtcgaaggaggcactcgtcgagggccggtccatcgaggacctgtgccttcgagacagcgcctggtacgacgacaacggcgccgaactgtggttgggagagcgcgtggtcgggctcgacccgacagactcggtcgtgaggctggcgtccggttccgaaatcgggtttgaccgtctcctcctcgcgccgggcgtcgaaccgattcggcttcccgtaccgggcagtgagcttgccggcgtgcactacctgcgcacctacgacgacgcggtccagctccggcacgccgtggaggtccgaggccgtccgtgccgtgtggtcgtcgtgggcggcggcttcatcggatcggagctggccgcgtcgctcggcgcgatgggtgcgcttgtgacggtcgtggaggcaacctcccagttgatggtgcaggcgctcggggaggaagtgggtgccctcctcaccaggcgtcaccgccaggccgggatcgatgtgcggttggacgcgagggtcgagcgactcagcggggaaaccacagtgcagggagtccagctcgctgacggctccgaactgccctgcgacctggtggtggtcggcatcggcgccaagccccgtttggagtggctggagggctccggtgtggagctcgctgacgggatcgtcgtcgacgagcactgccgcacctcgcgggaaaacgtcttcggcgccggggatgcgacagtgatgtactccccgcgactgggccgccaccgccgggtcgagcacgaggccaacgcccaagcccaaggcgtcgtagcagcccgcaacatgctgggcggcaacgccgtccacgacccagtcgactactgctggtccatccagcacgacctcgacatctggacgctcggcgaaacgggccgcggtggggaggtgtcggtcgagatcggagacgggggcaagcacgcgctcgcgacgtatcgcctggccgggaatgtggtgggcgtcgtgggtatcaaccgtccagacgacctcgcgcccgccagggagctgctgacgtcgctgatcgcatag

SEQ ID NO: 24 - protein, 390 aa, Nocardiodes aromaticivorans
MRRHYEYLVVGGGVAGGRAVEALSKRADSVALVSAEHWRPYARPPLSKEALVEGRSIEDLCLRDSAWYDDNGAELWLGERVVGLDPTDSVVRLASGSEIGFDRLLLAPGVEPIRLPVPGSELAGVHYLRTYDDAVQLRHAVEVRGRPCRVVVVGGGFIGSELAASLGAMGALVTVVEATSQLMVQALGEEVGALLTRRHRQAGIDVRLDARVERLSGETTVQGVQLADGSELPCDLVVVGIGAKPRLEWLEGSGVELADGIVVDEHCRTSRENVFGAGDATVMYSPRLGRHRRVEHEANAQAQGVVAARNMLGGNAVHDPVDYCWSIQHDLDIWTLGETGRGGEVSVEIGDGGKHALATYRLAGNVVGVVGINRPDDLAPARELLTSLIA

SEQ ID NO: 25 - DNA, 1173 bp, Cycloclasticus zancles
atggatcaaagtgaaagaattttagtcaacgaagaggtcgtgaaaaagaacaagttatggccgaactttattaaggccaagctggggtttagaaaccattggtaccccgtcatgttcggtaaagaaatagaagagggcaagcctgttaaggcgatgttatgtggtgaaaacctattactcaaccgaattgatggaaaagtttatgccatcaaagataggtgtttacaccgaggtgttgccttttctaaaaagcctgagtgttatacaaaagagaccattacctgttggtatcatgcttggacgtatcgatgggacgatggctcgttatgcgacattatgacggaccctaaaagcgatatgattggtaagcatcgtcttaaaacgtataccgcgcaagaagcaaaagggcttgtatttatttttctcggcgatattgaacctacccctctgattaatgacgtaccacctgggtttttagatgaagggcgtgcgattagaggcattaaacgagaagttgggtcaaactggagaatcgctgctgaaaatggttttgattcaacacatgtttttattcataaagacagtaagttaataccaaacaatgagaccgttattccattagggtttgcgacagatcgtgaagaagaagcaaaaggcaccttatgggaagtagttaataacgaagacggacctaagggtgtttacgataatatcggccagcatgcggttcccgtcgttgagggtaaagtagatggtgaaacagttcttcgtccggttattggtggtgataaacgcatagccaaccaaatctcaatatggatgccaggcgcccttaaagtagacccctttccagacccttcattgattcaatttgaatggtatgtgccaagagatgaaaactcacattggtatattcaaacgcttggtaaagaagtggctaatgaagctgaagagcaagagtttgaaaaagattttaatgaaaagtgggaagactgggggctacgtggctttaatgatgatgatatttgggcccgtgaagcgatggaagagttttacaaggatgactggggttggattaaagaacagttatttgagccagatggaaatatagtcgcgtggagacagttagccagtgaagcaaatcgcggtgtccaaacactagaagatttataa

SEQ ID NO: 26 - protein, 390 aa, Cycloclasticus zancles
MDQSERILVNEEVVKKNKLWPNFIKAKLGFRNHWYPVMFGKEIEEGKPVKAMLCGENLLLNRIDGKVYAIKDRCLHRGVAFSKKPECYTKETITCWYHAWTYRWDDGSLCDIMTDPKSDMIGKHRLKTYTAQEAKGLVFIFLGDIEPTPLINDVPPGFLDEGRAIRGIKREVGSNWRIAAENGFDSTHVFIHKDSKLIPNNETVIPLGFATDREEEAKGTLWEVVNNEDGPKGVYDNIGQHAVPVVEGKVDGETVLRPVIGGDKRIANQISIWMPGALKVDPFPDPSLIQFEWYVPRDENSHWYIQTLGKEVANEAEEQEFEKDFNEKWEDWGLRGFNDDDIWAREAMEEFYKDDWGWIKEQLFEPDGNIVAWRQLASEANRGVQTLEDL

SEQ ID NO: 27 - DNA, 327 bp, Cycloclasticus zancles
atgtctgaattaatgatgctttgtaaaacagccgaggtgaccgaagatgcaccaattcaagtggtggtagatggtttgcccccactagcggtttatgagtttaataaaagttattacgttaccagtgatatttgcacacacggaatggcgtttatgacagagggtgaacaagatggaaatgaaattgagtgcccttttcatggtggtgcatttaattttgtcacaggggaagtggtgtctatgccctgtcatattccattagagacttttccagtcgtcataaatgatgaatacgtgtgtattgaaaaaccagtacttgagaaatga

SEQ ID NO: 28 - protein, 108 aa, Cycloclasticus zancles
MSELMMLCKTAEVTEDAPIQVVVDGLPPLAVYEFNKSYYVTSDICTHGMAFMTEGEQDGNEIECPFHGGAFNFVTGEVVSMPCHIPLETFPVVINDEYVCIEKPVLEK

SEQ ID NO: 29 - DNA, 1023 bp, Cycloclasticus zancles
atgacaagttataacgtaaaaatcagcggacaggaactggagtttgcttgtgaggaaggagatactattttgcgtgcagcactacgcgcaggtgtaggcatgccttacgaatgtaattcaggtggttgtggtgcctgtaaagttgaagtgttaaacggtgaagtggagaatatttgggaagatgcgccgggcctgtcacctcgtgatattaaaaaaggtcgcaaattgagctgtcaatgtattccaactgaagaccttgagattaaggttcgcttaaaccccgaagcgatgccgttacataagccaattaagggtaaggccgttttatttgaaataaataaactaacagaagatatggcagagttttgctttaaaacggagcatcctgctcattttaaagcagggcaatttgccttattagatttcccgggcattacaggctcacgtggttattcaatgtgtaacctgccaaatgaggaaggtgagtggcgttttattattaagaaaatgccagacggtagtgctaccacaactttatttgaagattatgaagtgggtgcggagattgtaatcgacgggccttatggtttggcatatttgaaaccagaaattccaagagatatcgtttgtgtgggtggtggttcaggcttgtcacccgagatgtcgatcattaaggcagctgccagagatcctcagctaagtgatagaaatatttatttgttctacggaggtcgtacaccaagtgatatttgtccgcctaagcttattgaagcagatgatgatttacgtggccgagtgaagaatttcaatgccgtatcagacgttgaagcagccgaagcagcagggtggaatggtgatgttgggtttatccatgagttgttaggaaaaacattgggagaaaaacttccagagcacgaattttatttctgtggccctcctcctatgacagatgctttaacacgtatgctgatgactgaatacaaagtgccgtttgatcaaattcattacgatcgtttttattaa

SEQ ID NO: 30 - protein, 340 aa, Cycloclasticus zancles
MTSYNVKISGQELEFACEEGDTILRAALRAGVGMPYECNSGGCGACKVEVLNGEVENIWEDAPGLSPRDIKKGRKLSCQCIPTEDLEIKVRLNPEAMPLHKPIKGKAVLFEINKLTEDMAEFCFKTEHPAHFKAGQFALLDFPGITGSRGYSMCNLPNEEGEWRFIIKKMPDGSATTTLFEDYEVGAEIVIDGPYGLAYLKPEIPRDIVCVGGGSGLSPEMSIIKAAARDPQLSDRNIYLFYGGRTPSDICPPKLIEADDDLRGRVKNFNAVSDVEAAEAAGWNGDVGFIHELLGKTLGEKLPEHEFYFCGPPPMTDALTRMLMTEYKVPFDQIHYDRFY

SEQ ID NO: 31 - DNA, 1146 bp, Pseudoxanthomonas spadix
atgacagcattggtttcccccgaagtgctcagccaagtcaagggctgggctcgctatgtcgaggcaaagctgggtttccgcaatcattggtatcccatccgctttgcccatgaggtcctggagcagacacccgtccctatcaagctgctgggtgaaaagatcttgctgaaccggatcgacggtaaggtctacgcgatcaaggaccggtgcttgcaccgtggagttgccttctcggacaaactcgaatgtctgacgaaagataccataagctgctggtatcacggctggacctaccgttgggacaccggcaagctggtcgatatcctcaccaatccccagagcatccagatcggacggcacaacgttcgctcctatccagtgcaggaggtgaagggcattgtgttcgtctacgtcggtgatcgggaaccgaccgatctgtctgaagacgtccctccgggatttcttgatgcagaccggacggtgttcgggttgcaccgtgaggtggcatccaactggcgtattgcagcggagaacgggttcgacgcaggacacgtttacatccacaaggattcgatcctgctgaaggggaacgacattgcccttccgctgggctttgcgcctggcagctccgcccagcttactcggtcagagatcgagccgggacgccccaaaggtgtcttcgacttgatcggcgagcattcggtgccagtgttcgaaggaacgatcgagggtgaggtgaagattcgcggcaacatgggcagcaagcgagtcgccgaaaacatctcgatgtggcttccgtgcgtgctacgggtcgagcccttccccaatccgggactcacgcaatacgagtggtacgtgccgattgacgaggacaatcacttgtatttccagaccatcggaaaactgtgtccgaccgaggctgagacggacgagttcaagcaagagttcgacgaaaagtgggtgtcgatggcgctgcacggcttcaacgacgacgacgtcatggcccgcctttcgacccagcggttctaccaggatgaccgtggctggatcaatgagattctctatgagccggataagtcgatcattgagtggcgccgtctggcatccgagcacaaccggggcatccagtcgcgcgagcacctctga

SEQ ID NO: 32 - protein, 381 aa, Pseudoxanthomonas spadix
MTALVSPEVLSQVKGWARYVEAKLGFRNHWYPIRFAHEVLEQTPVPIKLLGEKILLNRIDGKVYAIKDRCLHRGVAFSDKLECLTKDTISCWYHGWTYRWDTGKLVDILTNPQSIQIGRHNVRSYPVQEVKGIVFVYVGDREPTDLSEDVPPGFLDADRTVFGLHREVASNWRIAAENGFDAGHVYIHKDSILLKGNDIALPLGFAPGSSAQLTRSEIEPGRPKGVFDLIGEHSVPVFEGTIEGEVKIRGNMGSKRVAENISMWLPCVLRVEPFPNPGLTQYEWYVPIDEDNHLYFQTIGKLCPTEAETDEFKQEFDEKWVSMALHGFNDDDVMARLSTQRFYQDDRGWINEILYEPDKSIIEWRRLASEHNRGIQSREHL

SEQ ID NO: 33 - DNA, 321 bp, Pseudoxanthomonas spadix
atgatcgccgtgacctttctgagcgaggacggcacttcgcaggtccggagtgcagcgcttggcacgaccctcatgcgtattgctgtccagtcgggagttcagggaatcttggccgaatgcggtggggcctgcgcctgcgcgacctgccacgtgattgtggacgcaagttgggtcgccgcagccgggccggcgaacgatctggaaaacgagatgcttgactacgcagtcaaccgtcagcccggctctcgccttgcctgtcagatcgagctaactgaaagcatgaacgggctcatcgtgcgtataccaaaaactcagaaatga

SEQ ID NO: 34 - protein, 106 aa, Pseudoxanthomonas spadix
MIAVTFLSEDGTSQVRSAALGTTLMRIAVQSGVQGILAECGGACACATCHVIVDASWVAAAGPANDLENEMLDYAVNRQPGSRLACQIELTESMNGLIVRIPKTQK

SEQ ID NO: 35 - DNA, 987 bp, Pseudoxanthomonas spadix
atggaccccgttgcccgcacggtcagcacggacgatggaacctgccaagggttcgacgtgcttgtgatggctaccggtgcacggcctcgtgtgcttgatatccccgggtctactctggacggcgtcggcgcgctacgcacattagccgacgctgtcgcacttggtgccgcaagtccgcctggcgcccgcctcgtgatcctcggcggcgggacgatcggcctcgaagtcgccgcctccctccgcgccgcaggcgtgacggtgactgttgtcgagcgggcgcaccgtttgctcgcccgcaccgcgagcgaaaccatggctgcctggttgcgtactcggcacgaggcccagggcgtggtttttcatctcgggcgcaccgcagtcgagatcgaaggaaaggatcgcaaggtgtcagcggtcctgctcgatgacggcactcggatcgcatgcgatgccgttctgtcctgtgtgggcgtggagccggataccgggttggctgtgcaggccggactactccaccacggtcctatcaaagttgattcagccgcgcgggtctgtcctggtctctacgcgatcggcgactgcacgagcaggcctgtcaccggccatgagggcaatgttcgtctcgagagtgtgccaagcgccttagagcaggggcgccaggtcattgcggatctttgcggcagcgcgccaccacctctagaagtcccttggttctggtccgatcagtatgcttacaaactgcagactgcgggcctagtgccccagggggcggcgctcgttgtcagaacacgtccgggatccgaccgaatcacagtcgcgcatctcacgccttcagggcatctgctcgctgtcgaagcggtcggtgcgccgggcgattttctagcggcccggcaggttatgggccgcgctggaattctcgatccagatctgctgagcgatcccgcgataccgtttcgcgccgcttgcgtcgggatggtggcgcgatga

SEQ ID NO: 36 - protein, 328 aa, Pseudoxanthomonas spadix
MDPVARTVSTDDGTCQGFDVLVMATGARPRVLDIPGSTLDGVGALRTLADAVALGAASPPGARLVILGGGTIGLEVAASLRAAGVTVTVVERAHRLLARTASETMAAWLRTRHEAQGVVFHLGRTAVEIEGKDRKVSAVLLDDGTRIACDAVLSCVGVEPDTGLAVQAGLLHHGPIKVDSAARVCPGLYAIGDCTSRPVTGHEGNVRLESVPSALEQGRQVIADLCGSAPPPLEVPWFWSDQYAYKLQTAGLVPQGAALVVRTRPGSDRITVAHLTPSGHLLAVEAVGAPGDFLAARQVMGRAGILDPDLLSDPAIPFRAACVGMVAR

SEQ ID NO: 37 - DNA, 3585 bp, Paraburkholderia xenovorans
aagcttatgagttcagcaatcaaagaagtgcagggagcccctgtgaagtgggttaccaattggacgccggaggcgatccgggggttggtcgatcaggaaaaagggctgcttgatccacgcatctacgccgatcagagtctttatgagctggagcttgagcgggtttttggtcgctcttggctgttacttgggcacgagagtcatgtgcctgaaaccggggacttcctggccacttacatgggcgaagatccggtggttatggtgcgacagaaagacaagagcatcaaggtgttcctgaaccagtgccggcaccgcggcatgcgtatctgccgctcggacgccggcaacgccaaggctttcacctgcagctatcacggctgggcctacgacatcgccggcaagctggtgaacgtgccgttcgagaaggaagccttttgcgacaagaaagaaggcgactgcggctttgacaaggccgaatggggcccgctccaggcacgcgtggcaacctacaagggcctggtctttgccaactgggatgtgcaggcgccagacctggagacctacctcggtgacgcccgcccctatatggacgtcatgctggatcgcacgccggccgggactgtggccatcggcggcatgcagaagtgggtgattccgtgcaactggaagtttgccgccgagcagttctgcagtgacatgtaccacgccggcaccacgacgcacctgtccggcatcctggcgggcattccgccggaaatggacctctcccaggcgcagatacccaccaagggcaatcagttccgggccgcttggggcgggcacggctcgggctggtatgtcgacgagccgggctcactcctggcggtgatgggccccaaggtcacccagtactggaccgagggtccggctgccgagcttgcggaacagcgcctggggcacaccggcatgccggttcgacgcatggtcggccagcacatgacgatcttcccgacctgttcattcctgcccaccttcaacaacatccggatctggcacccgcgtggtcccaatgaaatcgaggtgtgggccttcaccctggtcgatgccgacgccccggcggagatcaaggaagaatatcgccggcacaacatccgcaacttctccgcaggcggcgtgtttgagcaggacgatggcgagaactgggtggagatccagaaggggctacgtgggtacaaggccaagagccagccgctcaatgcccagatgggcctgggtcggtcgcagaccggtcaccctgattttcctggcaacgtcggctacgtctacgccgaagaagcggcgcggggtatgtatcaccactggatgcgcatgatgtccgagcccagctgggccacgctcaagccctgataagctagcaaggagatataccatgacaaatccatccccgcattttttcaaaacatttgaatggccaagcaaggcggctggccttgagttgcagaacgagatcgagcagttctactaccgcgaagcgcagttgcttgaccaccgggcctacgaggcctggtttgccctgctggacaaagatatccactacttcatgccgctgcgcaccaatcgcatgatccgggagggcgagctggaatattccggcgaccaggatttagcccatttcgatgaaacccatgaaaccatgtacgggcgcatccgcaaggtgacctcggacgtgggctgggcggagaacccgccttcccgcacgcgccacctggtctccaacgtcatcgtcaaggagacggccacgccggataccttcgaggtcaattccgcattcatcctgtaccgcaatcggcttgagcgccaggtcgacatcttcgcgggcgaacgccgggacgtgctgcgccgcgccgacaacaaccttggtttcagcatcgccaagcgcaccatcctgctcgacgccagtaccttgctgtcgaacaacctgagcatgttcttctagtaagctagcaaggagatataccatgaaatttaccagagtttgtgatcgaagagatgtgcccgaaggcgaagccctgaaggtcgaaagtggaggcacctccgtcgcgattttcaatgtggatggcgagctgttcgcaacacaggaccgctgcacccacggcgactggtccctgtccgatggcggctatcttgaaggtgacgtggtggaatgctcactgcacatggggaagttttgcgttcgcacgggcaaggtcaaatcaccgccgccctgtgaggcactgaagatatttccgatccgcatcgaagacaatgacgtgctggtcgacttcgaagccgggtatctggcgccatgataagctagcaaggagatataccatgatcgacaccatcgccatcatcggcgccggcctggccggttcgacggctgcgcgcgcactgcgcgcccagggatacgaggggcgcatccacctgctcggggatgagtcgcatcaggcctatgaccggaccacgctgtccaagacggtgctggcgggcgagcagcccgagccgcctgcaatcctggacagcgcctggtacgcatcggcccatgtggatgtccagctcgggcgacgggtgagttgcctggatctggccaaccgccagattcagtttgaatcgggcgccccgctggcctacgaccggctgctgctggccaccggcgcgcgcgcccggcgcatggcgattcggggtggcgacctggcaggcatccataccttgcgagacctcgccgacagccaggcgctgcggcaggcgctgcaaccgggccagtcgctggtcatcgtcggcggaggcctgatcggttgcgaggtggcgaccaccgcccgcaagctgagtgtccatgtcacgattctggaagccggcgacgagttgctggtgcgcgtgctgggtcaccggaccggggcatggtgtcgggccgaactggaacgcatgggtgtccgcgtggagcgcaatgcacaggccgcgcgcttcgaaggccaggggcaggtgcgcgccgtgatctgcgccgacgggcgccgggtgcccgccgatgtggtcttggtcagcattggcgccgagccggcggacgagctggcccgtgccgctggcatcgcctgcgcgcgcggcgtgctggtcgacgccaccggcgccacctcgtgtccagaggtgttcgccgccggtgacgtcgccgcctggccgctgcgtcaagggggccagcgctcgctggagacctacctgaacagccagatggaggccgaaatcgcggccagcgccatgttgagtcagcccgtgccggcgccccaggtgccgacctcgtggacggagattgcaggccaccgcatccagatgattggcgatgccgaagggcccggcgagatcgtcgtacgcggcgacgcccagagcggccagccaatcgtgttgctcaggctgcttgatggctgcgtcgaggccgcgacggcgatcaatgccaccagggaattttctgtggcgacccgactggtcggcacccgggtttctgtttccgccgagcaactgcaggacgtcggctcgaacctgcgggatttactcaaagccaaaccgaattgataactcgag

SEQ ID NO: 38 - protein, 459 aa, Paraburkholderia xenovorans
MSSAIKEVQGAPVKWVTNWTPEAIRGLVDQEKGLLDPRIYADQSLYELELERVFGRSWLLLGHESHVPETGDFLATYMGEDPVVMVRQKDKSIKVFLNQCRHRGMRICRSDAGNAKAFTCSYHGWAYDIAGKLVNVPFEKEAFCDKKEGDCGFDKAEWGPLQARVATYKGLVFANWDVQAPDLETYLGDARPYMDVMLDRTPAGTVAIGGMQKWVIPCNWKFAAEQFCSDMYHAGTTTHLSGILAGIPPEMDLSQAQIPTKGNQFRAAWGGHGSGWYVDEPGSLLAVMGPKVTQYWTEGPAAELAEQRLGHTGMPVRRMVGQHMTIFPTCSFLPTFNNIRIWHPRGPNEIEVWAFTLVDADAPAEIKEEYRRHNIRNFSAGGVFEQDDGENWVEIQKGLRGYKAKSQPLNAQMGLGRSQTGHPDFPGNVGYVYAEEAARGMYHHWMRMMSEPSWATLKP

SEQ ID NO: 39 - DNA, 3558 bp, Sphingomonas sp. CB3
aagcttatgatcaagcgtccccctatcgatggctccgcgatggattcgttggaatccaggataagaaagcttgttcgccccgatgaaggggtgatccacgcttccgtttattccgaccccgagatttatcaactcgaactttcccgtatctttgcgcgatcgtggctgttgctttgtccggacagtcagattcccaacgctggcgattatttcgtcagctatatgggagaggatccggtcatcgtcgtccgccagcaagacggaacgatcgcggcgttcctcaaccagtgccgacaccgtggcggcgccctgtgccggggagagtccggcaacaccaagaatttcatctgcacctatcacgggtggacctatgacacgagcggaacgctgacgagcgttccattcgaagaggtcgtctacaaggcgccgctgaatcgcgcgaaatggagtgcccggcgtgtcccgcgtctggaggtgcatcacggcctcgtatttggttgctgggacgaggatgcgcccggttttcgtgaatcgctgggtgaagccgccgtatatttcgaccttaatttcggtcggaccgagggtgggctggcgacctacggcggcgtctataaatggcgggtgaaagccaattggaagctcgcggccgagcagttcacgaccgatgatttccatttcctgacttcgcattcttccgcgctgaccgcgctgactcctgaggatgcgccgccattctcgattgttcgcggtcgggtgttcacgagttcgaaggggcacgggggcggcttcctcatggaacgcgacagctttgccacagcgctcgccacgacaacgggccaggcagccagtaactacatgatggaggtcgagcttccgacggtcgagcagcggtatggcgaagcgatggccaatgccacgccgacctttgcgaacttctttccttcgaccggatatctccatgccaacaggacgctgcgttcgtggattccacgcggtcccgacgaaatggagatctgggcttggacgcttttcgaccgcggctcaccggacgaattgatggaagagagggcgaagataacggcgatgactttcggcccggccgggatctttgaacaggacgataccgccaactgggtggacgttcagcgcccgcttggcggcgcgatcgcacggcggacgaaactcaacatgcagatgggcgagccgacttcgttcgagggttggccggggatgacgggtttcgacagtagcgaattccccgcccgcaacttctactcgcgatggctccagcttctgagcacgccaaaccacgcgcttgaagctagcccgaccgacgacgaggagtgcagccatgtccgttgataagctagcaaggagatataccatgtccgttgaacccgtggctctcgatatgccagctatcgctgaggagcctggtcctcgactgcagtgggagatcgagcagttcctatatgctgaagccggccttctcgatgaccggcgttttgaggactggctcgcgttgatggctgacgacgtcgtctaccagatgccgcttcggacagaccggattcgccgggacgagcggcgtctcaaggccattgccgaagaggtcaagatattcgacgataatctcgaacgtcttcggacccgggtgaagcgtctccggtctggaaccgcctggtcagacgatccgcgcgctcgggtccggcacctgatctcgaatgtccagatctctaggggtcaacagcccgaggaaatcgaagtgatctccgttttcctcgtctacgtgtcgcggatggacgaagagcctacgctcttctccggccagcgccacgacgtcttgaggagcgatgccaatggcggctggaagatcgcccgacgcgtcgtgatcggcgatcagtccgtcattccctcgaacaacctgacgttgttcttctgataagctagcaaggagatataccatgcgctggattgacgccggcggagccgctgagctcgatgtcgacgaggtcgccaaattcgacgccgatgtcgggcccttggccatctaccataccgacggcggctatttcgcgacccaggatacctgcacgcatgctgtcgcttctctctccgacgggttcgtcgaagacgggatgatcgaatgcccgttgcacgcggcgaagttctgcatccgtactggaaaggccaagagcctgcccgctacggagccgttggagacttatcccgtacaggtcgtggatggccgaattctcgttggtctcccgcttgaactcggagccgaggcgtgataagctagcaaggagatataccatgatcggaagcgtcgcaatcgttggcgccagcgctgctggtgtcgctgccgccacgacgctgcgggacgaaggttatgagggcgagatcaccctcatcggcggcgagaccgacctgccatatgagcgaccggcggtatccaaggatattctcctgacgggcgcggcgccgccgatcattcctgaacagcgctacgccgaactgaacatcaagcttctcttgggaaccagggcggagcgcatcgacgcacgatacggccagatcgagctgagcgacgggcggacgatggtcagtgacaggctcctgctggcaaccggcggttggccgcggcgtttacccgtgcctggcgcggaattgggcggactgcattatgttcgggatgcgcgggatggacaggccatacggtccggtctgcggcccggcgcgcgtatcgccgttgtgggcggcggcctaatcggtgcggaagtggcggccagcgcggttcaggcgggctgcgaagtggactggatcgaagcggaaggactatgcttggcccgggcgctctcgcgtccgctggccgaggcgatgatggacgttcatcggcagcgtggggttcgcgtccacgccaatgcgcttgtcgtccgcctgatcggagagcgatccgtccaggcggtcgagcttgcggatggccgccggatcgacgccgatatggtcgttgtcggaatagggataacccccgccgccgaactggctgaggaagcagatctgacggtcagcgacgggatcgtgatcgaccccttttgtcgcacctcggccgagaacgtctatgccgccggagacgtcgcgcggcatcagacccgatatatggctacgccttcgcgactggaacactggcgcaacgcgcaggaacagggcgtcacggctgcaagggccatgttgggacatcggcagccctatgacgagctgccctggttctggacggatcaatatgacctgcacatcgaaggctgtggggtgatgcgcgccgatgatgaaaccatcctgcgcggcaatctcgccgatggcaacgccaccgtgtttcatctgcgcgccggaagcctcgtaggggcctgcgcgctgaacaggcagggtgatgtgcgtggagcgatgcgtttgatcacaagggggctgaccccgtcggccgacattctctcggacccgacgaaggatttgcgcaaaatcgaaaaggaactctcccgtgcctcagcttgataataactcgag

SEQ ID NO: 40 - protein, 451 aa, Sphingomonas sp. CB3
MIKRPPIDGSAMDSLESRIRKLVRPDEGVIHASVYSDPEIYQLELSRIFARSWLLLCPDSQIPNAGDYFVSYMGEDPVIVVRQQDGTIAAFLNQCRHRGGALCRGESGNTKNFICTYHGWTYDTSGTLTSVPFEEVVYKAPLNRAKWSARRVPRLEVHHGLVFGCWDEDAPGFRESLGEAAVYFDLNFGRTEGGLATYGGVYKWRVKANWKLAAEQFTTDDFHFLTSHSSALTALTPEDAPPFSIVRGRVFTSSKGHGGGFLMERDSFATALATTTGQAASNYMMEVELPTVEQRYGEAMANATPTFANFFPSTGYLHANRTLRSWIPRGPDEMEIWAWTLFDRGSPDELMEERAKITAMTFGPAGIFEQDDTANWVDVQRPLGGAIARRTKLNMQMGEPTSFEGWPGMTGFDSSEFPARNFYSRWLQLLSTPNHALEASPTDDEECSHVR

SEQ ID NO: 41 - DNA, 3645 bp, Terrabacter sp. YK3
aagcttatgctgactgtgaatgacagtggtcaactggtgagcccgaacgggcagacacctcaggcaccacctgtgaatcccgccctgtcgtctcagctcaaggaactgtccgagagcgagggtggcctgctggaccggcgcatgtttttcgaccctgagatctacaaggttgaacttgagcgcgtctttgcacgatcatggtcctttctctgccatgaaagccagctggccaaggccggggacttcttctcgacctacatcggcgccgatcccgtcgtggtgacccgacagcgcgacggatcgatcagcgcggtgctcaactcttgtcgccatcgtgggatgaaggtctgccgcgccgactgggggaacgcgaaggccttcacctgcacgtaccacggttggtcgtacagcacggatggctcgttggtgagcgtgccccgcgaggaatacgcctactacaacgagatcgacaagtcgaagttgggattgctgcgggttccacaggtgcagtcctacaaagggctggttttcggttgcttcgatcccgaagcgccgtcgcttgtcgacttcttgggcgacatgacctactacttggacatcctgcttgaccgtgtggatggcggcaccgaagtcatctccggtgtccacaagtggaagatgcggggcaactggaagcttgccgccgagcagttcagtggagacaactaccacaccatctccagccatatatcggtgctgctgtctgagttcccgcccgaggcggcggacgccttcgtgaatatcgacgggctcgagatcaacccagcggaaggccatggtattggtgttatgtactcgccgaccggagcgccgttctcggcggggagcagcgaggcgatcctgcgctggcgcgacgagacgcgccaggagtccatcaaccgccttggtaaggagcgcgtagaggggatgtcctggacgcacgccaacgtgttccccaacttctcttacctccacgacagctcggtcctgcgcgtttggatgcccaagagtcccaccgagatggaggcctggtcgtggtgcatcgtcgacaagaaggctccgcaggaggtgaagaatgcttggcgcacgcaggccatccgacacttcagccccggtggcacttgggaacaggacgacggcgagaactggagttactgctcaggtgctgggggtcaggagggagtggtgacccgactctccaagttgcatgtcgagatgggagtgggacacgagcgctcgcatccgacgctgcccggcaaggtcagtcacacctacagtgagcagaaccagcgcagtctgtaccgacgctgggccgagttcatggcggcggagtcttggaaggacatctccgtgccggtgcgtacgaccgaggtaatcgaccgaagcgacatggcgaaggcgggagaatcctgataagctagcaaggagatataccatgagcgttcttgagaatacgaatacagaggttattgacgtcgcccgtgcggtcgagaagttctactacaaggaggcgcgactccttgacgacaggctcttcacggagtggctcacattgtgggccgacgatgcccacctgtgggcgcctctccggtataacctgtctcggcgggagcagcagttcgagtattccggtgaagacgacttcggatacttcgacgacgacaaaccgaatctcgagaagcgggtgcgggggttggagaccgggcaggcgtgggccgaggatcccccgacgcgcaccaggcgcctcattacgaacgtcgaagtggagtcggacgattccggtgtaggagactaccgggcccggtcccacttcctcgtctatcgcaaccgcatggaagccgatgttgacctgcacgctggatgtcggcgcgacatcctccgccggactgccacggacggtctgctcatcgcccgccgcgaggtcatcctagacaacaacgtgttgctgtctaggaatctgagcatcttcttctgataagctagcaaggagatataccatgaccaacaacgacgtggaagtggcgctgccgaacgtcgagggccgcacatggcgccgtgcctgtgcggcccacgacgtgcccgaggacgaaggtctgtgcgtcggtacgctgccgcccgtctcggtgtttgtaacagagggcgagtacttctgtatcgacgatacgtgcacccacgagacctactcgttggcggacgggtgggtcgcggacggtttcgtcgaatgcgccctccacctcgctaagttcaacttgcgcaccggcgagccgctcgcgccgcccgccacgacggctgtggccgtccatcccgtcgcactcgtcgacggggtcctttatgttgcgcttccgagcgcgtacctcatcaaggagtgataagctagcaaggagatataccatgaccgcaccgcaccacgtcatcgtcggtggcagtgctgccggtgtcgcagcggcactagccatgcggagaaatggcttcgagggtcggatcactctcgtggaagcagcctccgaggagccctacgagcgaccgcctctgtcgaagtctttcaccgaccttgacgcgccgcgtcggatcctcccaccgagcacgtacgtcgaggaagacatcgacctgctgctcggcatgccggtcgcagcgctcgatgtcgaccggaaggtggtgcggttgcctgacggcgagggactcggggcggatgccgtgctagtggcgaccggtgtcaacgctcggcgtctgggagttccgggagaatatctcgagcatgtcctggtgctgcgtggcctggcggatgcacgtgcgctggcggcgcgcctcgacgtgggcggtccttgggtgatcgtcggaggagggttcatcggcctcgaggcggcggccgtcgcgcggggaagagggatcgatgtcacggtagtcgaggcgatgccggtgccgctggccggcgtgctgggccctgcccttgcagcccacgtccagcggatgcacgagcgtgagggggtgcggattctgggggggcgcactgtgaccgagttcgtgggggagagggaggtcgagaaggtcgtcctggacgatggctcggttctggatgcggccaccgtactcgttggctgcggggtggagcccaacgacgagctggcccgagacgcaggggtgtactgcaacggcggcatcgtcgcggaccgtcacggtcgcacgagtgtcccctggatctgggcggccggcgacgtcgccaccttcgtcagtccgttcaccgggcgtcgccagcgcatcgagcactgggacgtcgccaatcgtctaggcacagtcaccggagccaacatggttggggtaccggcagtcaacacagatgcgccgtacttctggtccgatcaatacggacatcggctccagatgtatggccgacaccagccaggcgaccagttcgtcgtccgacctggcgtgaccacggcgcagttcgtcgcattctgggtccgcgatgggcgggtcaccgcggcggctgcgatcgactcgccgaaggagttgcgggcgaccaagccactgatcgagggacgagttcccgttatggcatcggacctgatcgacccggccgtctcattgcgtgcgctcgggcgtgtcgctcatccatgataataactcgag

SEQ ID NO: 42 - protein, 474 aa, Terrabacter sp. YK3
MLTVNDSGQLVSPNGQTPQAPPVNPALSSQLKELSESEGGLLDRRMFFDPEIYKVELERVFARSWSFLCHESQLAKAGDFFSTYIGADPVVVTRQRDGSISAVLNSCRHRGMKVCRADWGNAKAFTCTYHGWSYSTDGSLVSVPREEYAYYNEIDKSKLGLLRVPQVQSYKGLVFGCFDPEAPSLVDFLGDMTYYLDILLDRVDGGTEVISGVHKWKMRGNWKLAAEQFSGDNYHTISSHISVLLSEFPPEAADAFVNIDGLEINPAEGHGIGVMYSPTGAPFSAGSSEAILRWRDETRQESINRLGKERVEGMSWTHANVFPNFSYLHDSSVLRVWMPKSPTEMEAWSWCIVDKKAPQEVKNAWRTQAIRHFSPGGTWEQDDGENWSYCSGAGGQEGVVTRLSKLHVEMGVGHERSHPTLPGKVSHTYSEQNQRSLYRRWAEFMAAESWKDISVPVRTTEVIDRSDMAKAGES

SEQ ID NO: 43 - DNA, 2312 bp, Unknown
  Naphthalene-catabolic genes from oil- contaminated soil
aagcttatgacagtaaagtggattgaagcagtcgctctttctgacatccttgaaggtgacgtcctcggcgtgactgtcgagggcaaggagctggcgctgtatgaagttgaaggcgaaatctacgctaccgacaacctgtgcacgcatggttccgcccgcatgagtgatggttatctcgagggtagagaaatcgaatgccccttgcatcaaggtcggtttgacgtttgcacaggcaaagccctgtgcgcacccgtgacacagaacatcaaaacatatccagtcaagattgagaacctgcgcgtaatgattgatttgagctaataagctagcaaggagatataccatgaattacaataataaaatcttggtaagtgaatctggtctgagccaaaagcacctgattcatggcgatgaagaacttttccaacatgaactgaaaaccatttttgcgcggaactggctttttctcactcatgatagcctgattcctgcccccggcgactatgttaccgcaaaaatggggattgacgaggtcatcgtctcccggcagaacgacggttcgattcgtgcttttctgaacgtttgccggcatcgtggcaagacgctggtgagcgtggaagccggcaatgccaaaggttttgtttgcagctatcacggctggggcttcggctccaacggtgaactgcagagcgttccatttgaaaaagatctgtacggcgagtcgctcaataaaaaatgtctggggttgaaagaagtcgctcgcgtggagagcttccatggcttcatctacggttgcttcgaccaggaggcccctcctcttatggactatctgggtgacgctgcttggtacctggaacctatgttcaagcattccggcggtttagaactggtcggtcctccaggcaaggttgtgatcaaggccaactggaaggcacccgcggaaaactttgtgggagatgcataccacgtgggttggacgcacgcgtcttcgcttcgctcgggggagtctatcttctcgtcgctcgctggcaatgcggcgctaccacctgaaggcgcaggcttgcaaatgacctccaaatacggcagcggcatgggtgtgttgtgggacggatattcaggtgtgcatagcgcagacttggttccggaattgatggcattcggaggcgcaaagcaggaaaggctgaacaaagaaattggcgatgttcgcgctcggatttatcgcagccacctcaactgcaccgttttcccgaacaacagcatgctgacctgctcgggtgttttcaaagtatggaacccgatcgacgcaaacaccaccgaggtctggacctacgccattgtcgaaaaagacatgcctgaggatctcaagcgccgcttggccgactctgttcagcgaacgttcgggcctgctggcttctgggaaagcgacgacaatgacaatatggaaacagcttcgcaaaacggcaagaaatatcaatcaagagatagtgatctgctttcaaaccttggtttcggtgaggacgtatacggcgacgcggtctatccaggcgtcgtcggcaaatcggcgatcggcgagaccagttatcgtggtttctaccgggcttaccaggcacacgtcagcagctccaactgggctgagttcgagcatgcctctagtacttggcatactgaacttacgaagactactgatcgctaataagctagcaaggagatataccatgatgatcaatattcaagaagacaagctggtttccgcccacgacgccgaagagattcttcgtttcttcaattgccacgactctgctttgcaacaagaagccactacgctgctgacccaggaagcgcatttgttggacattcaggcttaccgtgcttggttagagcactgcgtggggtcagaggtgcaatatcaggtcatttcacgcgaactgcgcgcagcttcagagcgtcgttataagctcaatgaagccatgaacgtttacaacgaaaattttcagcaactgaaagttcgagttgagcatcaactggatccgcaaaactggggcaacagcccgaagctgcgctttactcgctttatcaccaacgtccaggccgcaatggacgtaaatgacaaagagctacttcacatccgctccaacgtcattctgcaccgggcacgacgtggcaatcaggtcgatgtcttctacgccgcccgggaagataaatggaaacgtggcgaaggtggagtacgaaaattggtccagcgattcgtcgattacccagagcgcatacttcagacgcacaatctgatggtctttctgtgataataactcgag

SEQ ID NO: 44 - protein, 104 aa, Unknown
  Naphthalene-catabolic genes from oil- contaminated soil
MTVKWIEAVALSDILEGDVLGVTVEGKELALYEVEGEIYATDNLCTHGSARMSDGYLEGREIECPLHQGRFDVCTGKALCAPVTQNIKTYPVKIENLRVMIDLS

SEQ ID NO: 45 - DNA, 3534 bp, Rhodococcus opacus
aagcttatgctgagcaacgaactccggcagaccctccaaaagggtttgcatgacgtgaattccgactggaccgtcccggccgcgatcatcaacgatccagaggtgcacgacgtcgagcgcgagcggatctttggtcatgcgtgggttttcctcgcgcatgagagtgagatccccgagcgcggtgactacgttgtgcggtacatctccgaagatcagttcattgtctgccgcgacgagggcggtgagatccgcggtcacctcaatgcttgccgccaccgcggtatgcaggtgtgccgcgcggagatggggaacacctcacacttccgatgcccttaccacggttggacctacagcaacacgggaagtctggtcggtgttccggccggcaaggatgcgtatggcaatcagctgaagaaatccgactggaacctacggccgatgccgaatctggccagctacaagggcctgatcttcggctcgctggacccgcatgccgattcgctcgaggactacctcggcgacctgaagttctacctcgatattgttctggaccgcagtgacgccggactgcaggtcgtcggcgcgccgcagcgttgggtgatcgacgcgaactggaagctcggtgccgacaactttgtcggcgacgcgtatcacaccatgatgacccaccgctcgatggtcgagctggggctcgccccgcccgacccgcagttcgcgctctatggcgaacacatccacaccgggcacgggcacggcctgggtatcattggtccgccgccgggtatgccgttgccggagttcatgggccttccggagaacatcgttgaagagttggaacgtcggctcacgccggagcaggtcgaaatcttccggcccactgccttcatccatggcaccgtgttcccgaatctatcgatcggcaacttcctgatggggaaggatcacctctctgcgccgactgcattcctgacgctgcgcctctggcatccgctcggaccggacaagatggaggtgatgtctttcttcctcgtggagaaggacgcacccgattggttcaaggacgagagctataagtcctacctgcgcaccttcggaatctccggcggcttcgaacaggacgacgccgagaactggcgcagcatcacccgtgttatgggcggccagttcgccaagaccggggaactcaactatcagatgggccgcggcgttctcgaacccgatccgaactggaccggaccgggagaggcctacccactggactacgccgaggctaaccagcgcaacttcctcgaatactggatgcagctcatgctcgcggagtcaccgctgcgcgacggcaacagcaacggcagtggcacggcggacgcgtcgaccccggcggcagctaagtccaagtccccagctaaagcggaggcgtagtaagctagcaaggagatataccatgaatacgcagacacgggtctcggacaccaccgttcgagagatcaccgaatggctctacatggaggcagagctgctcgacgccgggaagtaccgggagtggctggcactcgtcaccgaggatctgagctacgttgtgccgattcgggtcacccgggaacgtgaggccgtgaccgacgtcgtcgagggaatgacccatatggacgacgacgcggactcgatggagatgcgcgtgctgcgcctcgagaccgagtacgcgtgggcggaggatccgccgtcgcgttcacggcacttcgtcaccaacgttcgggtcgctacgggtgatagtgaggacgagttcaaggtcacctcgaacctgctgctctaccgcacccgcggtgacgttgctacatacgatgtcctctcgggcgagcgtacggatgtcctccggcgcgcaggcgatagcttcctgatggccaaacgtgttgtgctgctagatcagacaacaatcatgacacacaacctcgccctgattatgtgataagctagcaaggagatataccatgaagactctcatcgcaacggaagagacgcaggctgacccggcaaccgagctgtgggtctgcgaggtctgtgaagacgtgtacgaccccaggctgggcgacccggagggtggcatctccccaggaactgccttccaggacatccccgacgattgggtctgtccggtctgcggggcacgcaagaaggaattccgcaagctcaggccgggcgaggagtaccagtacgttggcgaagacctcgtgacaggcgagctgggatgataagctagcaaggagatataccatgacagcggtcagcgagcccgacacacgcaccgtcgtgatcgtgggcacgggcatcgccggttccggtgccgcgcaggccctgcgcaaggaagggttcggcggcagcatcatcctgatcggcagcgaacctgaggagccgtaccgccgcccagcgctgtcgaaggagctactgtccgggaaagcgtcgatcgatcgggctcggttgcggccgtcgactttctggaccgagcagggtatcgatcttcggatcggcgcaactgtcacgagtatcgacacagattcccgcacagtacttttagccgacggtgacagcatcgactacgacgttctgattcttgccacgggtggacggtcccgacggttggagaacgaagattccgagcgcgttcactatcttcgggatatcgcagacatgcgacgcttgcaatcccagctgatcgaaggatcctcgcttttggtggtcggcggtggcttgatcggatcggaggtggcgtcaacggcacgcgacttgggttgcagtgtgcaggttctcgaagcgcaaccggtgcccctgtccaggctgcttccaccgtcgatagcggagaagatcgccgcgctgcacgcctcggcgggcgtcgccttgcagacgggagtcgacctcgagacgctcacgacgggtgccgacggcgtcaccgcacgtgcgcgtgacggacgcgagtggacagcggacttggccgtcgtcgcaatcggatccttgcccgataccgatgtggctgctgcggcgggtattgcggtggacaacgggatttcggtagacggatacctccggacctccgtcgttgatgtgtacgcgatcggcgatgtggccaacgtgcccaacggttttctcggcggcatgcaccgtggtgagcactggaacaccgcgcaggaccacgcagttgcagttgccaagaccatcgtcgggaaggaagaacccttcgaatccgtcccttggagttggtcgaaccaattcggccgcaacattcaagtagctggttggccaggcgcggacgacaccgtgattgttcgaggagacttggactcctatgacttcactgcgatctgcatgcgcgacggaaatatcgtcggtgctgtgagcgtgggccggccgaaggacattcgtgccgtccgaacccttatcgaacgctccccggacatcagcgccgacgtactcgccgatacaaacagggatctgaccgaacttgcggcgggtcttgtcgcctcaccggtgctctgataataactcgag

SEQ ID NO: 46 - protein, 470 aa, Rhodococcus opacus
MLSNELRQTLQKGLHDVNSDWTVPAAIINDPEVHDVERERIFGHAWVFLAHESEIPERGDYVVRYISEDQFIVCRDEGGEIRGHLNACRHRGMQVCRAEMGNTSHFRCPYHGWTYSNTGSLVGVPAGKDAYGNQLKKSDWNLRPMPNLASYKGLIFGSLDPHADSLEDYLGDLKFYLDIVLDRSDAGLQVVGAPQRWVIDANWKLGADNFVGDAYHTMMTHRSMVELGLAPPDPQFALYGEHIHTGHGHGLGIIGPPPGMPLPEFMGLPENIVEELERRLTPEQVEIFRPTAFIHGTVFPNLSIGNFLMGKDHLSAPTAFLTLRLWHPLGPDKMEVMSFFLVEKDAPDWFKDESYKSYLRTFGISGGFEQDDAENWRSITRVMGGQFAKTGELNYQMGRGVLEPDPNWTGPGEAYPLDYAEANQRNFLEYWMQLMLAESPLRDGNSNGSGTADASTPAAAKSKSPAKAEA

SEQ ID NO: 47 - DNA, 3411 bp, Nocardiodes sp. KP7
aagcttatgtcggtagtcagcggggataggaacatccaacggctcatctcgagcgggcggcagagcgtcgagaaggggcagttgcccgcccggctcgtggccaacgcggagattcacgagctggaagcggagcgggttttcggccggtcgtgggtgttcctcgcgcacgagtcggaggtgccggaggctggcgactatgtcgtgcgctacatgggcgacgactcggtgatcgtggtccgggacgagagcggctcggtgcgcgcgatggcgaactcgtgtcggcaccgcggcaccttgttgtgccggaccgaggcgggcaacacctcgcattttcgctgcccataccacggctggacctacaagaacaccggtgacctgaccggcgtacccgcgcaggaggaggtttacggcgcctcgatggacaaggcgcagtggaacctgacaccggtaccgcggctcgagtcctacaacggcctggtcttcggttgtctggacgacgcagcgccgacgctggtcgagtacctgggcgacatggcctggtacatcgacctgttcaccaagcgcagcgccggcggtctcgaggtccgcggcgagccgcagcgctgggtgatcgatgcgaactggaagctcggcgccgacaacttcgtcggcgacgcctaccacacgctgatgacgcaccgctcgatggcggagctcggtctcgtgccgccggacccgaacttcgcctccgcgccggcccacatcagcctgtcgggcggtcacggcctcggcgtcctcggcgctccgcccggctacgagatgccgccgttcatgaactacccggaggagatgatcgagggtctcgccgccagctacgggaaccagacgcacgtcgacgttttggagcggacgaccttcattcacgggacggtgttccccaacctgtcctttctcaacgtcatgatcagcaaggaccacatgtcggttcccgtcccgatgttgaccatgcgtctgtggcgcccgctcagccacgacacgatggaggtctggtcgtggttcctcatcgagcgggacgcaccggatgacttcaaggacctgtcctacgagacctatatccgcaccttcggggtgtccggggtgtttgagcaggacgacgccgagacctggcgatcgatcactaaggcgacaaagggtctgctcagtggtagccagcggctgaacttcgagatggggctgaacgtgctcggccgcgaccccgactggaagggccccggtcgcgcgctgtcgagcgggtacgccgagcagaaccagcgggagttctggggacggtggctcgagctcctcgaggacgccgacgacgagagcgctgtcttatgataagctagcaaggagatataccatgctgactactgttgacgagaatctgatgctccgtcttgccgtcgaggactttttcttcacagaatcggccctgctggacgacggacgtttccgggaatggctggacctggtgaccgaggacatcaagtacgtcatcccggtccagacgacgcgggaacgcgcgcatggggggagcgccagctcgacgaccatggcgcactgggacgacgactacaccgggctggagatgcgcatcctccggctcgacaccgagtacgcgtgggccgaggacccgccgtcgaagctgcggcatttcgtctccaacgtgcgcgtccgacccggctccggggccgacgagtacgaggtgcgttcgaacgtgatggtgtcgcgtagccgcggcgacagcaggaccacagagctgctcacggccgagcgtcaggacgtgctgcgtcggaccgaccagggcttccgcctcgcccggcgcaccgtcgtcctcgatcacgtcgtgatcgccacgcacaatcttgcgttcttcttctagtaagctagcaaggagatataccatgcgtgtggatgttgacccacagcggtgctgcggctaccggctctgcgtcgagaccgcgccggatgtcttccagatcaacgcgatcgggaaggccgtcgtcgcactcgaccccatcccgaccgagcggcacgacgctgtccgcgcggcagcgcgcgagtgtcccggggccgcgatcacgctgagcgacgacctaggccccgctcagtgataagctagcaaggagatataccatgacgggaggccaggtggcggcgctgatcggcggtgggatggcgggcgtgcacgccgccgaggtactgcgccgggacggcttcgacgggcgggtgctgctggtctccgcggagcagcacctgccctacgaccgcccgccgctatccaaggcgctcctccgcggcgagctggccctcgcggactgcctgctgcgcccaccggagtggtacgaggaacaggggatcgaggtgttgctcggcgtctcggtggacgctctcgaccccggtcggcgcacgctccggctcagcaccggcgagcaggtcgagttcgaccgcgcgctgctcgcgaccggagcccgtccgcggtggccgctcggcctcgcgccggggtgtggcccggtgttcgcgctgcgcaccgtcgacgactgcctggccatccggtcgcgactgcggtcgggcgcgtcggtggtggttgtcggcggcggattcgtcggcgccgagctcgcctccagcgcggcgtcgctgggctgccgggtcacgatgctggaagcggccgacgcgccgttccagcgcgtactggggcggaccgttggcgagttgttcgggagattctacgccactggagggatccggctcgtcaccggcgtgcaggtcaccgggacgagcgtgggtccggagggcgcgcgcctcaccgcgggcgacggccggttctgggacgccgacgtcgtcgtcgtcggcgtcggtgtcgtgccgaacaccgagctggcggtcgacgccgggctgcgggtgtcggacggcgtcgaggtggacgcgtactgcacgacatcggcaccgcacgtcttcgctgcgggcgacgtcgccaaccgtcccgaccccgtcctaggccgccgggtccggatcgaacactggcagaacgcccagcaccagggcaccgccgccgggcgggccatgctcggcatccgggaacccttcgacggggtgccctggttctggtccgaccagttcggcctgaacctgcaggtcgccggcttccccgaccgggccgaccgggtcgtcgtccgaggccgcctcgaaggggaccggttcgccgccttctaccttgccggcccgacgttggtcgcggcgctgggtgtgggttgcgcgggggaggtgcacctgagccggcggctgatcgccgcccgggcgcacgtcgatccccagcggctcaccgacgagcacagcgacctgcgcgatgcgctcctggcgtccgacgtaccgacggcatgataataactcgag

SEQ ID NO: 48 - protein, 449 aa, Nocardiodes sp. KP7
MSVVSGDRNIQRLISSGRQSVEKGQLPARLVANAEIHELEAERVFGRSWVFLAHESEVPEAGDYVVRYMGDDSVIVVRDESGSVRAMANSCRHRGTLLCRTEAGNTSHFRCPYHGWTYKNTGDLTGVPAQEEVYGASMDKAQWNLTPVPRLESYNGLVFGCLDDAAPTLVEYLGDMAWYIDLFTKRSAGGLEVRGEPQRWVIDANWKLGADNFVGDAYHTLMTHRSMAELGLVPPDPNFASAPAHISLSGGHGLGVLGAPPGYEMPPFMNYPEEMIEGLAASYGNQTHVDVLERTTFIHGTVFPNLSFLNVMISKDHMSVPVPMLTMRLWRPLSHDTMEVWSWFLIERDAPDDFKDLSYETYIRTFGVSGVFEQDDAETWRSITKATKGLLSGSQRLNFEMGLNVLGRDPDWKGPGRALSSGYAEQNQREFWGRWLELLEDADDESAVL

(19) United States
(12) Patent Application Publication
(10) Pub. No.: US 2019/0178066 A1
(43) Pub. Date: Jun. 13, 2019

(54) BIOLOGICAL UPGRADING OF HYDROCARBON STREAMS WITH DIOXYGENASES

(71) Applicant: ExxonMobil Research and Engineering Company, Annandale, NJ (US)

(72) Inventors: Zarath M. Summers, High Bridge, NJ (US); David O. Marler, Easton, PA (US); Jay B. Patel, Princeton, NJ (US); Katherine G. Landuyt, Dallas, TX (US)

(21) Appl. No.: 16/186,837

(22) Filed: Nov. 12, 2018

Related U.S. Application Data
(60) Provisional application No. 62/597,512, filed on Dec. 12, 2017.

(51) Int. Cl. E21B 43/16; C12N 9/02; C09K 8/58; C12N 15/52; C10G 32/00

(52) U.S. Cl. CPC E21B 43/16; C12N 9/0071; C09K 8/58; C12N 15/52; C10G 2300/202; C10G 32/00; C12Y 114/12012; C12Y 118/01002; C10G 2300/206; C12N 9/0095

(57) ABSTRACT

Dioxygenases and methods of biologically upgrading hydrocarbon streams, such as crude oil, using dioxygenases are provided herein. The dioxygenases can be used to remove impurities such as metals, heteroatoms, or asphaltenes from a hydrocarbon stream. In some cases, the dioxygenases can be chemically or genetically modified and can be used in different locations such as petroleum wells, pipes, reservoirs, tanks and/or reactors.

### CROSS-REFERENCE TO RELATED APPLICATIONS

This application claims priority to U.S. Provisional Application No. 62/597,512 filed Dec. 12, 2017 which is herein incorporated by reference in its entirety. This application is related to two other co-pending U.S. provisional applications filed on Dec. 12, 2017: U.S. Provisional Application Nos. 62/597,488 and 62/597,502, each of which is herein incorporated by reference in its entirety.

### REFERENCE TO A SEQUENCE LISTING

This application contains references to amino acid sequences and/or nucleic acid sequences which have been submitted concurrently herewith as the sequence listing text file entitled “62027102_1.txt”, file size 113 KiloBytes (KB), created on 29 Jun. 2017. The aforementioned sequence listing is hereby incorporated by reference in its entirety pursuant to 37 C.F.R. § 1.52(e)(5).

### FIELD

The present disclosure relates to dioxygenases and methods of using dioxygenases for upgrading hydrocarbon streams, for example, crude oil.

### BACKGROUND

This section provides background information related to the present disclosure. The references cited in this section are not necessarily prior art.

Typically, any number of hydrocarbon streams, such as whole crude, diesel, hydrotreated oils, atmospheric gas oils, vacuum gas oils, coker gas oils, atmospheric and vacuum residues etc., may require removal of heteroatom species, such as nitrogen-containing and/or sulfur-containing species. In particular, increasing supplies of crude oils with higher nitrogen and sulfur content paired with increasing regulations on sulfur content of refined products has resulted in the need for additional means of heteroatom removal. Catalytic hydrotreating and/or adsorption can be used to lower content of nitrogen-containing and/or or sulfur-containing species from hydrocarbon feeds. However, nitrogen-containing species can poison the hydrotreating catalysts. Thus, high pressure and high temperature hydrotreating is necessary to overcome nitrogen poisoning of the catalysts and to effectively remove the sulfur-containing species to meet sulfur content specifications of the various feeds, which can result in increased costs and emissions from refineries.

Hydrocarbon streams can also include various metal species, such as vanadium and nickel, which require removal because the presence of such metals can be detrimental to refining processes. For example, metals can be particularly damaging to catalytic cracking and catalytic hydrogenation units as they can be deposited on the catalysts rendering them inactive. Nickel and vanadium, which can be abundantly found in crude oil, can be the most damaging during catalytic refining processes. However, nickel and vanadium can be very difficult to remove as they most commonly exist as oil-soluble metalloporphyrins. Chemical, thermal and physical methods have traditionally been used for metals removal. Some chemical methods include use of a demetallization agent complexation and acid treatments (sulfuric, hydrofluoric, hydrochloric). Some thermal methods include visbreaking, coking, and hydrogenation and favored physical methods include distillation and solvent extraction. Unfortunately, these methods have inherent limitations. For example, chemical and thermal processing can require severe operating conditions, cause extensive side reactions, introduce product contamination, generate lower value products, and consume energy and fuel. With regard to physical methods, distillation alone can be non-selective, fail to provide complete metals removal, and solvent extraction can decrease the yield of desired hydrocarbon.

Thus, there is a need for improved methods for selectively removing impurities, such as heteroatoms and metals. Especially needed are methods which can remove heteroatoms and/or metals from hydrocarbons that leave the hydrocarbon backbone untouched, unlike some adsorption techniques. Removal of the entire hydrocarbon molecules is undesirable because up to 10 wt % of some crudes can contain heteroatoms and a 10 wt % loss of hydrocarbons is not economically feasible.

U.S. 2016/0333307 to Fong et al. reports using hydrogen sulfide:NADP+oxidoreductase, hydrogen sulfide:ferredoxin oxidoreductase, sulfide:flavocytochrome-c oxidoreductase, sulfide:quinone oxidoreductase, sulfur dioxygenase, sulfite oxidase, or combinations thereof to remove sulfur from fuel.

U.S. 2016/0160105 to Dhulipala et al. reports sulfhydrylases or cysteine synthases added to fuels—including fuel wells—to remove sulfur.

U.S. 2011/0089083 to Paul et al. reports using globins, peroxidases, pyrrolases, and cytochromes to remove metals from fuel.

U.S. Pat. No. 5,624,844 to Xu et al. reports using oxygenases to remove metals from fuel.

WO 2008/058165 reports immobilizing enzymes on substrates for use in catalyzing chemical reactions.

D'Antonio & Ghiladi (2008) report in an abstract from the 60^{th} Southeast Regional Meeting of the American Chemical Society that oxygenases might be used to demetallize petroporphyrins in crude oil.

### SUMMARY

This section provides a general summary of the disclosure, and is not a comprehensive disclosure of its full scope or all of its features.

The present disclosure provides dioxygenases, for example having at least 40% sequence identity to any one or more of SEQ ID NOs: 2, 8, 14, 20, 26, 32, 38, 40, 42, 44, 46, and 48, to upgrade the quality of hydrocarbon streams. Compositions comprising a dioxygenase for upgrading hydrocarbon streams are also provided herein.

Also disclosed herein are recombinant or modified dioxygenase enzymes, in which the enzyme has been made more hydrophobic than its native counterpart. In certain embodiments, the dioxygenase is hydrophobically modified to be at least 10% more enriched in hydrophobic amino acids selected from the group consisting of Ala, Gly, Ile, Leu, Met, Pro, Phe, and Trp. In certain embodiments, additional hydrophobic amino acids are added to the enzyme. In certain embodiments, amino acids with polar or charged side chains are replaced with hydrophobic amino acids. In certain embodiments the dioxygenase is treated chemically (e.g., dioxygenase is rinsed with n-propanol, dioxygenase is conjugated to a polyethylene glycol, or disulfide bridges are added to the dioxygenase) to be more hydrophobic.

Methods of biologically upgrading hydrocarbon streams, such as crude oil, are additionally disclosed herein. These methods involve contacting the hydrocarbon stream with an enzyme and/or composition described herein. In certain embodiments, the contacting occurs while the hydrocarbon streams are moved through pipes or stored in reservoirs or tanks. In certain embodiments, the contacting occurs while the hydrocarbon streams are present in a reactor. In certain embodiments, the contacting occurs before the hydrocarbon stream, e.g., crude oil, may be extracted from the earth, for example by sending the enzymes and/or compositions described herein into a petroleum well. In certain embodiments, the contacting results in the removal of impurities (e.g., metal, heteroatoms, or asphaltenes) from the hydrocarbon stream.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments, and not all possible implementations. The drawings and their corresponding descriptions are not intended to limit the scope of the present disclosure.

FIG. 1 shows the percentage of initial carbazole that is converted into more refined product by the various E. coli strains indicated.

FIG. 2 shows the percentage of initial dibenzothiophene that is converted into more refined product by the various E. coli strains indicated.

FIG. 3 shows the percentage of initial dibenzofuran that is converted into more refined product by the various E. coli strains indicated.

FIG. 4 shows the percentage of initial fluorene that is converted into more refined product by the various E. coli strains indicated.

FIG. 5 shows a flow chart illustrating an exemplary process for selecting and using enzymes to purify less refined fuel sources.

### DETAILED DESCRIPTION

Unless defined otherwise, all technical and scientific terms used herein have the same meaning as commonly understood by one of ordinary skill in the art to which this invention belongs. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference. In case of conflict between definitions incorporated by reference and definitions set out in the present disclosure, the definitions of the present disclosure will control.

Although methods and materials similar or equivalent to those described herein can be used in practice or testing of the present invention, suitable methods and materials are described below. The materials, methods and examples are illustrative only and are not intended to be limiting. Other features and advantages of the invention will be apparent from the detailed description and from the claims.

### Definitions

To facilitate an understanding of the present invention, a number of terms and phrases are defined below.

As used in the present disclosure and claims, the singular forms “a,” “an,” and “the” include plural forms unless the context clearly dictates otherwise.

Wherever embodiments are described herein with the language “comprising,” otherwise analogous embodiments described in terms of “consisting of” and/or “consisting essentially of” are also provided.

The term “and/or” as used in a phrase such as “A and/or B” herein is intended to include “A and B,” “A or B,” “A,” and “B.”

As used herein, and unless otherwise specified, the term “Cₙ” means hydrocarbon(s) having n carbon atom(s) per molecule, wherein n is a positive integer.

As used herein, the term “hydrocarbon(s)” means a class of compounds containing hydrogen bound to carbon, which may be linear, branched or cyclic, and encompasses (i) saturated hydrocarbon compounds, (ii) unsaturated hydrocarbon compounds, and (iii) mixtures of hydrocarbon compounds (saturated and/or unsaturated) including mixtures of hydrocarbon compounds having different values of n. The term “hydrocarbon(s)” is also intended to encompass hydrocarbons containing one or more heteroatoms, such as, but not limited to nitrogen, sulfur, and oxygen, and/or containing one or more metals, such as vanadium and nickel. Non-limiting examples of heteroatom-containing and metal-containing hydrocarbons include porphyrins or petroporphyrins, and metalloporphyrins. The term “porphyrin” refers to a cyclic structure typically composed of four modified pyrrole rings interconnected at their a carbon atoms via methane bridges (═C—) and having two replaceable hydrogens on two nitrogens, where, for example, various metal atoms can be substituted to form a metalloporphyrin. Examples of nitrogen-containing species include, but are not limited to carbazoles, imidazoles, pyrroles, quinones, quinilines and combinations thereof. Examples of sulfur-containing species include, but are not limited to mercaptans, thiols, disulfides, thiophenes, benzothiophenes, dibenzothiophenes and combinations thereof. Examples of oxygen-containing species include, but are not limited to furans, indoles, carbazoles, benzcarbazoles, pyridines, quinolines, phenanthridines, hydroxypyridines, hydroxyquinolines, dibenzofuranes, naphthobenzofuranes, phenols, aliphatic ketones, carboxylic acids, and sulfoxides.

As used herein, the term “hydrocarbon stream” refers to any stream comprising hydrocarbons, which may be present in the oil reservoir/wellbore, pipes, tanks, reactors, etc. Examples of hydrocarbon streams include, but are not limited to hydrocarbon fluids, whole crude oil, diesel, kerosene, virgin diesel, light gas oil (LGO), lubricating oil feedstreams, heavy coker gasoil (HKGO), de-asphalted oil (DAO), fluid catalytic cracking (FCC) main column bottom (MCB), steam cracker tar, streams derived from crude oils, shale oils and tar sands, streams derived from the Fischer-Tropsch processes, reduced crudes, hydrocrackates, raffinates, hydrotreated oils, atmospheric gas oils, vacuum gas oils, coker gas oils, atmospheric and vacuum residues (vacuum resid), deasphalted oils, slack waxes and Fischer-Tropsch wax. The hydrocarbon streams may be derived from various refinery units, such as, but not limited to distillation towers (atmospheric and vacuum), hydrocrackers, hydrotreaters and solvent extraction units.

As used herein, the term “asphaltene” refers to a class of hydrocarbons, present in various hydrocarbon streams, such as crude oil, bitumen, or coal, that are soluble in toluene, xylene, and benzene, yet insoluble in paraffinic solvents, such as n-alkanes, e.g., n-heptane and n-pentane. Asphaltenes may be generally characterized by fused ring aromaticity with some small aliphatic side chains, and typically some polar heteroatom-containing functional groups, e.g., carboxylic acids, carbonyl, phenol, pyrroles, and pyridines, capable of donating or accepting protons intermolecularly and/or intramolecularly. Asphaltenes may be characterized as a high molecular weight fraction of crude oils, e.g., an average molecular weight (about 1000 and up to 5,000) and very broad molecular weight distribution (up to 10,000), and high coking tendency.

As used herein, the term “upgrade” or “upgrading” generally means to improve quality and/or properties of a hydrocarbon stream and is meant to include physical and/or chemical changes to a hydrocarbon stream. Further, upgrading is intended to encompass removing impurities (e.g., heteroatoms, metals, asphaltenes, etc.) from a hydrocarbon stream, converting a portion of the hydrocarbons into shorter chain length hydrocarbons, cleaving single ring or multi-ring aromatic compounds present in a hydrocarbon stream, and/or reducing viscosity of a hydrocarbon stream.

As used herein, the term “hydrophobic” refers to a substance or a moiety, which lacks an affinity for water. That is, a hydrophobic substance or moiety tends to substantially repel water, is substantially insoluble in water, does not substantially mix with or be wetted by water or to do so only to a very limited degree and/or does not absorb water or, again, to do so only to a very limited degree.

The term “heterologous” with regard to a gene regulatory sequence (such as, for example, a promoter) means that the regulatory sequence or is from a different source than the nucleic acid sequence (e.g., protein coding sequence) with which it is juxtaposed in a nucleic acid construct. By way of non-limiting example, a slyD gene from E. coli is heterologous to a slyD promoter from Y. pestis. Similarly, the slyD gene is heterologous to the hypB promoter, even when both slyD and hypB are from E. coli.

The term “expression cassette,” as used herein, refers to a nucleic acid construct that encodes a protein or functional RNA (e.g. a tRNA, a short hairpin RNA, one or more microRNAs, a ribosomal RNA, etc.) operably linked to expression control elements, such as a promoter, and optionally, any or a combination of other nucleic acid sequences that affect the transcription or translation of the gene, such as, but not limited to, a transcriptional terminator, a ribosome binding site, a splice site or splicing recognition sequence, an intron, an enhancer, a polyadenylation signal, an internal ribosome entry site, etc.

The term “operably linked,” as used herein, denotes a configuration in which a control sequence is placed at an appropriate position relative to the coding sequence of a polynucleotide sequence such that the control sequence directs the expression of the coding sequence of a polypeptide and/or functional RNA). Thus, a promoter is in operable linkage with a nucleic acid sequence if it can mediate transcription of the nucleic acid sequence. When introduced into a host cell, an expression cassette can result in transcription and/or translation of an encoded RNA or polypeptide under appropriate conditions. Antisense or sense constructs that are not or cannot be translated are not excluded by this definition. In the case of both expression of transgenes and suppression of endogenous genes (e.g., by antisense, or sense suppression) one of ordinary skill will recognize that the inserted polynucleotide sequence need not be identical, but may be only substantially identical to a sequence of the gene from which it was derived. As explained herein, these substantially identical variants are specifically covered by reference to a specific nucleic acid sequence.

“Naturally-occurring” and “wild-type” (WT) refer to a form found in nature. For example, a naturally occurring or wild-type nucleic acid molecule, nucleotide sequence, or protein may be present in, and isolated from, a natural source, and is not intentionally modified by human manipulation.

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window. The degree of amino acid or nucleic acid sequence identity can be determined by various computer programs for aligning the sequences to be compared based on designated program parameters. For example, sequences can be aligned and compared using the local homology algorithm of Smith & Waterman (1981) Adv. Appl. Math. 2:482-89, the homology alignment algorithm of Needleman & Wunsch (1970) J Mol. Biol. 48:443-53, or the search for similarity method of Pearson & Lipman (1988) Proc. Nat'l. Acad. Sci. USA 85:2444-48, and can be aligned and compared based on visual inspection or can use computer programs for the analysis (for example, GAP, BESTFIT, FASTA, and TFASTA in the Wisconsin Genetics Software Package, Genetics Computer Group, 575 Science Dr., Madison, Wis.).

The BLAST algorithm, described in Altschul et al. (1990) J Mol. Biol. 215:403-10, is publicly available through software provided by the National Center for Biotechnology Information (at the web address www.ncbi.nlm.nih.gov). This algorithm identifies high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra.). Initial neighborhood word hits act as seeds for initiating searches to find longer HSPs containing them. The word hits are then extended in both directions along each sequence for as far as the cumulative alignment score can be increased. Cumulative scores are calculated for nucleotides sequences using the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below due to the accumulation of one or more negative-scoring residue alignments; or the end of either sequence is reached. For determining the percent identity of an amino acid sequence or nucleic acid sequence, the default parameters of the BLAST programs can be used. For analysis of amino acid sequences, the BLASTP defaults are: word length (W), 3; expectation (E), 10; and the BLOSUM62 scoring matrix. For analysis of nucleic acid sequences, the BLASTN program defaults are word length (W), 11; expectation (E), 10; M=5; N=−4; and a comparison of both strands. The TBLASTN program (using a protein sequence to query nucleotide sequence databases) uses as defaults a word length (W) of 3, an expectation (E) of 10, and a BLOSUM 62 scoring matrix. See, Henikoff & Henikoff (1992) Proc. Nat'l. Acad. Sci. USA 89:10915-19.

In addition to calculating percent sequence identity, the BLAST algorithm also performs a statistical analysis of the similarity between two sequences (see, e.g., Karlin & Altschul (1993) Proc. Nat'l. Acad. Sci. USA 90:5873-87). The smallest sum probability (P(N)), provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, preferably less than about 0.01, and more preferably less than about 0.001.

“Pfam” is a large collection of protein domains and protein families maintained by the Pfam Consortium and available at several sponsored World Wide Web sites. Pfam domains and families are identified using multiple sequence alignments and hidden Markov models (HMMs). Pfam-A families, which are based on high quality assignments, are generated by a curated seed alignment using representative members of a protein family and profile hidden Markov models based on the seed alignment, whereas Pfam-B families are generated automatically from the non-redundant clusters of the latest release of the Automated Domain Decomposition algorithm (ADDA; Heger A, Holm L (2003) J Mol Biol 328(3):749-67). All identified sequences belonging to the family are then used to automatically generate a full alignment for the family (Sonnhammer et al. (1998) Nucleic Acids Research 26: 320-322; Bateman et al. (2000) Nucleic Acids Research 26: 263-266; Bateman et al. (2004) Nucleic Acids Research 32, Database Issue: D138-D141; Finn et al. (2006) Nucleic Acids Research Database Issue 34: D247-251; Finn et al. (2010) Nucleic Acids Research Database Issue 38: D211-222).

The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. et al., (1979) Principles of Protein Structure, Springer-Verlag). According to such analyses, groups of amino acids can be defined where amino acids within a group exchange preferentially with each other, and therefore resemble each other most in their impact on the overall protein structure (Schulz, G. E. et al., (1979) Principles of Protein Structure, Springer-Verlag). Examples of amino acid groups defined in this manner include an “aromatic or cyclic group,” including Pro, Phe, Tyr, and Trp. Within each group, subgroups can also be identified. For example, the group of charged amino acids can be sub-divided into sub-groups including: the “positively-charged sub-group,” comprising Lys, Arg and His; and the “negatively-charged sub-group,” comprising Glu and Asp. In another example, the aromatic or cyclic group can be sub-divided into sub-groups including: the “nitrogen ring sub-group,” comprising Pro, His, and Trp; and the “phenyl sub-group” comprising Phe and Tyr. In another further example, the hydrophobic group can be sub-divided into sub-groups including: the “large aliphatic non-polar sub-group,” comprising Val, Leu, and Ile; the “aliphatic slightly-polar sub-group,” comprising Met, Ser, Thr, and Cys; and the “small-residue sub-group,” comprising Gly and Ala. Examples of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn such that a free —NH₂ can be maintained.

### Dioxygenases

As disclosed herein, dioxygenases, particularly enzyme class EC1.14.12 dioxygenases also known as 1,2-hydroxylating naphthalene, NADH:oxygen oxidoreductase, but referred to herein simply as “dioxygenase” for simplicity, can be used to upgrade hydrocarbon streams. By contacting a hydrocarbon stream (e.g., crude oil) with a dioxygenase, impurities such as, heteroatoms, metals and asphaltenes can be removed and properties of the hydrocarbon stream can be improved, for example, viscosity may be lowered. Additionally, the fraction of the upgraded product that is recoverable can be increased. In certain embodiments, the dioxygenase is capable of cleaving heteroatom-carbon bonds (e.g., nitrogen-carbon bonds, sulfur-carbon bonds) and carbon-carbon bonds in non-porphyrin compounds. Examples of non-porphyrin compounds include, but are not limited to pyridine, pyrrole, indole, acridine, carbazole, dibenzothiophene, dibenzofuran, fluorene, phenanthrene, anthracene, tetracene, chrysene, triphenylene, pyrene, pentacene, benzo(a)pyrene, corannulene, benzo(ghi)perylene, coronene, ovalene, benzo(c)fluorine, other polyaromatic hydrocarbons, and any of the listed compounds with substitutions.

In certain embodiments, the dioxygenase can be a dioxygenase that classifies as belonging to subfamily cd08881. In certain embodiments, the dioxygenase classifies as belonging to Pfam family PFAM00848 or PFAM11723. Although the enzyme(s) can be present in the context of a host cell (e.g., a microbial cell), in certain embodiments the enzymes are substantially free or even totally free of cells, cell components, or cellular debris beyond the bare enzyme itself.

In some embodiments, the dioxygenase may be thermally stable from about 15° C. to about 150° C., about 50° C. to about 120° C. or about 90° C. to about 120° C.

In certain embodiments, the dioxygenase has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:2.

In certain embodiments, the dioxygenase has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:8.

In certain embodiments, the dioxygenase has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:14.

In certain embodiments, the dioxygenase has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:20.

In certain embodiments, the dioxygenase has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:26.

In certain embodiments, the dioxygenase has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:32.

In certain embodiments, the dioxygenase has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:38.

In certain embodiments, the dioxygenase has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:40.

In certain embodiments, the dioxygenase has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:42.

In certain embodiments, the dioxygenase has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:44.

In certain embodiments, the dioxygenase has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:46.

In certain embodiments, the dioxygenase has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:48.

### Hydrophobic Modification

In certain embodiments, dioxygenases as described herein can be modified to become more hydrophobic. Because the hydrocarbon stream may be a hydrophobic environment, by making the enzyme (in particular those enzyme surfaces that are exposed to the hydrophobic environment of the hydrocarbon stream) more hydrophobic, the enzyme can be better able to tolerate the stresses of the environment.

In certain embodiments, the enzymes can be modified to be more hydrophobic by the inclusion of a greater number of hydrophobic amino acids (Ala, Gly, Ile, Leu, Met, Pro, Phe, and Trp) in the enzyme's primary sequence. This can be accomplished in a number of different ways, none of which are mutually exclusive of each other. For example, one can replace a given polar (Asn, Cys, Gln, Ser, Thr, and Tyr) or charged (Arg, Asp, Glu, His, and Lys) amino acid with a hydrophobic amino acid. Additionally or alternatively, one can add one or more additional hydrophobic amino acids between two amino acids already present in the primary sequence of the wild type. Additionally or alternatively, one can add one or more (e.g., at least 5, at least 10, at least 20, at least 30, at least 40, or at least 50) additional hydrophobic amino acids at the amino and/or carboxy terminus of the enzyme. The result of these additions and/or substitutions can result in an enzyme that is at least 5% (e.g., at least 10%, at least 15%, at least 20%, at least 25%, at least 30%, at least 35%, at least 40%, at least 45%, or at least 50%) more hydrophobic than the corresponding wild-type enzyme sequence.

In order for an enzyme's amino acid sequence to be modified relative to the corresponding wild type sequence, the modified sequence must be less than 100% identical to its corresponding wild type sequence. In certain embodiments, the modified enzyme is no more than about 95% identical to the corresponding wild type, for example no more than about 90%, no more than about 85%, no more than about 80%, no more than about 75%, no more than about 70%, no more than about 65%, or no more than about 60% identical. However, the modified enzyme will still be at least about 40% (for example, at least 50%, at least 60%, at least 70%, at least 80%, at least 90%, at least 91%, at least 92%, at least 93%, or at least 94%) identical to the corresponding wild type sequence (e.g., a sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, and 48).

Additionally or alternatively, in certain embodiments an enzyme (e.g., a dioxygenase) can be made more hydrophobic by chemical modification. In certain embodiments, the enzyme can be rinsed with n-propanol. In certain embodiments polyethylene glycol can be conjugated to the enzyme. In certain embodiments, disulfide bridges can be added to the enzyme. The addition of disulfide bridges can affect the enzyme's tertiary structure. Therefore additional disulfide bridges must be placed carefully. The person of ordinary skill knows how to place disulfide bridges in a manner that will cause minimal disruption to enzymatic (e.g., dioxygenase) activity.

### Nucleic Acids

Also described herein are nucleic acids encoding dioxygenases and other enzymes for use with the methods and compositions described herein. The person of ordinary skill knows that the degeneracy of the genetic code permits a great deal of variation among nucleotides that all encode the same protein. For this reason, it is to be understood that the representative nucleotide sequences disclosed herein are not intended to limit the understanding of phrases such as “a nucleotide encoding a protein having at least 70% identity to SEQ ID NO . . . ” or “a construct encoding SEQ ID NO . . . ”.

In certain embodiments, the nucleotide encodes a dioxygenase having at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) identity to a sequence selected from the group consisting of SEQ ID NOs: 2, 8, 14, 20, 26, 32, 38, 40, 42, 44, 46, and 48. In certain embodiments, the nucleotide is selected from the group consisting of SEQ ID NOs:1, 7, 13, 19, 25, 31, 37, 39, 41, 43, 45, and 47.

In certain embodiments, the nucleotide encodes a ferredoxin having at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) identity to a sequence selected from the group consisting of SEQ ID NOs:4, 10, 16, 22, 28, and 34. In certain embodiments, the nucleotide is selected from the group consisting of SEQ ID NOs:3, 9, 15, 21, 27, and 33.

In certain embodiments, the nucleotide encodes a ferredoxin reductase having at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) identity to a sequence selected from the group consisting of SEQ ID NOs:6, 12, 18, 24, 32, and 36. In certain embodiments, the nucleotide is selected from the group consisting of SEQ ID NOs:5, 11, 17, 23, 31, and 35.

In certain embodiments, the nucleotides disclosed herein are incorporated into expression cassettes. The choice of regulator elements such as promoter or terminator or splice site for use in expression cassettes depends on the intended cellular host for gene expression. The person of ordinary skill knows how to select regulatory elements appropriate for an intended cellular host. A large number of promoters, including constitutive, inducible and repressible promoters, from a variety of different sources are well known in the art. Representative sources include for example, viral, mammalian, insect, plant, yeast, and bacterial cell types, and suitable promoters from these sources are readily available, or can be made synthetically, based on sequences publicly available on line or, for example, from depositories such as the ATCC as well as other commercial or individual sources. Promoters can be unidirectional (i.e., initiate transcription in one direction) or bi-directional (i.e., initiate transcription in both directions off of opposite strands). A promoter may be a constitutive promoter, a repressible promoter, or an inducible promoter. Non-limiting examples of promoters include, for example, the T7 promoter, the cytomegalovirus (CMV) promoter, the SV40 promoter, and the RSV promoter. Examples of inducible promoters include the lac promoter, the pBAD (araA) promoter, the Tet promoter (U.S. Pat. Nos. 5,464,758 and 5,814,618), and the Ecdysone promoter (No et al. (1996) Proc. Natl. Acad. Sci. 93:3346-51).

In certain embodiments, the nucleotides and/or expression cassettes disclosed herein can be incorporated into vectors. A vector can be a nucleic acid that has been generated via human intervention, including by recombinant means and/or direct chemical synthesis, and can include, for example, one or more of: 1) an origin of replication for propagation of the nucleic acid sequences in one or more hosts (which may or may not include the production host); 2) one or more selectable markers; 3) one or more reporter genes; 4) one or more expression control sequences, such as, but not limited to, promoter sequences, enhancer sequences, terminator sequences, sequence for enhancing translation, etc.; and/or 5) one or more sequences for promoting integration of the nucleic acid sequences into a host genome, for example, one or more sequences having homology with one or more nucleotide sequences of the host microorganism. A vector can be an expression vector that includes one or more specified nucleic acid “expression control elements” that permit transcription and/or translation of a particular nucleic acid in a host cell. The vector can be a plasmid, a part of a plasmid, a viral construct, a nucleic acid fragment, or the like, or a combination thereof.

In certain embodiments the nucleotide coding sequences may be revised to produce messenger RNA (mRNA) with codons preferentially used by the host cell to be transformed (“codon optimization”). Thus, for enhanced expression of transgenes, the codon usage of the transgene can be matched with the specific codon bias of the organism in which the transgene is desired to be expressed. The precise mechanisms underlying this effect are believed to be many, but can include the proper balancing of available aminoacylated tRNA pools with proteins being synthesized in the cell, coupled with more efficient translation of the transgenic mRNA when this need is met. In some examples, only a portion of the codons is changed to reflect a preferred codon usage of a host microorganism. In certain examples, one or more codons are changed to codons that are not necessarily the most preferred codon of the host microorganism encoding a particular amino acid. Additional information for codon optimization is available, e.g. at the codon usage database of GenBank. The coding sequences may be codon optimized for optimal production of a desired product in the host organism selected for expression. In certain examples, the nucleic acid sequence(s) encoding a dioxygenase, ferredoxin, or ferredoxin reductase is/are codon optimized for expression in E. coli. In some aspects, the nucleic acid molecules of the invention encode fusion proteins that comprise an enzyme (e.g., a dioxygenase). For example, the nucleic acids of the invention may comprise polynucleotide sequences that encode glutathione-S-transferase (GST) or a portion thereof, thioredoxin or a portion thereof, maltose binding protein or a portion thereof, poly-histidine (e.g. His6), poly-HN, poly-lysine, a hemagglutinin tag sequence, HSV-Tag, and/or at least a portion of HIV-Tat fused to the enzyme-encoding sequence.

The vector can be a high copy number vector, a shuttle vector that can replicate in more than one species of cell, an expression vector, an integration vector, or a combination thereof. Typically, the expression vector can include a nucleic acid comprising a gene of interest operably linked to a promoter in an expression cassette, which can also include, but is not limited to, a localization peptide encoding sequence, a transcriptional terminator, a ribosome binding site, a splice site or splicing recognition sequence, an intron, an enhancer, a polyadenylation signal, an internal ribosome entry site, and similar elements.

In certain embodiment, the expression cassettes or vectors disclosed herein comprise a nucleotide according to SEQ ID NOs: 13, 15, or 17, operably linked to a heterologous nucleotide sequence. Also contemplated as being within the scope of the present disclosure are variants of SEQ ID NO:13 that comprise such substitutions as to result in a nucleotide that encodes a protein sequence having at least 70% (for example, at least 75%, at least 80%, at least 85%, at least 90%, at least 95%, at least 96%, at least 97%, at least 98%, or at least 99%) identity to SEQ ID NO:14. Also contemplated as being within the scope of the present disclosure are variants of SEQ ID NO:15 that comprise such substitutions as to result in a nucleotide that encodes a protein sequence having at least 70% (for example, at least 75%, at least 80%, at least 85%, at least 90%, at least 95%, at least 96%, at least 97%, at least 98%, or at least 99%) identity to SEQ ID NO:16. Also contemplated as being within the scope of the present disclosure are variants of SEQ ID NO:17 that comprise such substitutions as to result in a nucleotide that encodes a protein sequence having at least 70% (for example, at least 75%, at least 80%, at least 85%, at least 90%, at least 95%, at least 96%, at least 97%, at least 98%, or at least 99%) identity to SEQ ID NO:18.

### Expression in Host Cells

In a further aspect, a recombinant microorganism or host cell, such as a recombinant E. coli, comprising a non-native gene encoding a dioxygenase is disclosed herein. In certain embodiments, the dioxygenase comprises an amino acid sequence having at least about 40% sequence identity to a sequence selected from the group consisting of SEQ ID NOs: 2, 8, 14, 20, 26, 32, 38, 40, 42, 44, 46, and 48, and/or to an active fragment of any thereof. For example, the non-native gene can encode a dioxygenase having an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a sequence selected from the group consisting of SEQ ID NOs: 2, 8, 14, 20, 26, 32, 38, 40, 42, 44, 46, and 48. In certain embodiments, the sequence having at least about 40% identity to a sequence selected from the group consisting of SEQ ID NOs: 2, 8, 14, 20, 26, 32, 38, 40, 42, 44, 46, and 48 is modified as described herein to make the resulting protein more hydrophobic than its wild-type counterpart.

In certain embodiments, the host cell can be a prokaryotic host cell, either gram negative or gram positive. By way of non-limiting example, the host cell can be an E. coli host cell. The skilled artisan is familiar with the media and techniques necessary for the culture of prokaryotic host cells, including E. coli.

In certain embodiments, the host cell can be a eukaryotic host cell, such as a yeast (e.g., S. cerevisiae or S. pombe) or an insect cell (e.g., an Spodoptera frugiperda cell such as Sf9 or Sf21). The skilled artisan is familiar with the media and techniques necessary for the culture of eukaryotic host cells, including yeast and insect cells.

### Additional Components

Nam et al. (2002) Appl. & Environ. Microbiol. 68(12):5882-90 have shown that EC1.14.12 dioxygenases are encoded in the Pseudomonas resinovorans genome in an operon with ferredoxin and ferredoxin reductase. These three enzymes (dioxygenase, ferredoxin, and ferredoxin reductase) function in a pathway together to metabolize carbazole. Therefore, a composition is also provided herein comprising a dioxygenase as described herein and a ferredoxin and/or a ferredoxin reductase which can be used to biologically upgrade hydrocarbon streams, for example by removing metals and/or heteroatoms.

In some embodiments, the ferredoxin and/or ferredoxin reductase may be thermally stable from about 15° C. to about 150° C., about 50° C. to about 120° C. or about 90° C. to about 120° C.

In certain embodiments, the ferredoxin has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:4.

In certain embodiments, the ferredoxin has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:10.

In certain embodiments, the ferredoxin has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:16.

In certain embodiments, the ferredoxin has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:22.

In certain embodiments, the ferredoxin has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:28.

In certain embodiments, the ferredoxin has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:34.

In certain embodiments, the ferredoxin reductase has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:6.

In certain embodiments, the ferredoxin reductase has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:12.

In certain embodiments, the ferredoxin reductase has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:18.

In certain embodiments, the ferredoxin reductase has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:24.

In certain embodiments, the ferredoxin reductase has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:30.

In certain embodiments, the ferredoxin reductase has at least 40% (for example, at least 50%, at least 60%, at least 70%, at least 80%, at least 90%, at least 91%, at least 92%, at least 93%, at least 94%, at least 95%, at least 96%, at least 97%, at least 98%, or at least 99%) sequence identity to SEQ ID NO:36.

In certain embodiments, a composition may comprise both a dioxygenase and a ferredoxin; or both of a dioxygenase and a ferredoxin reductase; or both of a ferredoxin and a ferredoxin reductase; or all three of a dioxygenase, a ferredoxin, and a ferredoxin reductase.

Additionally, one, two or more dioxygenases can be present in a composition, optionally with or without ferredoxins and/or ferredoxin reductases, and optionally including a nickel-binding protein or other enzyme to assist upgrading a hydrocarbon stream.

In addition to comprising other enzymes, a composition herein can comprise one or more of a lubricant, a surfactant, a viscosity additive, a fluid loss additive, a foam control agent, a weighting material, and a salt.

### Methods of Use

Also provided herein are methods of using the dioxygenases and compositions described herein. In various aspects, methods of biologically upgrading a hydrocarbon stream are provided herein comprising contacting the hydrocarbon stream with a dioxygenase and/or a composition described herein, for example, an EC1.14.12 dioxygenase. In some embodiments, the upgrading can comprise removing at least a portion of impurities from the hydrocarbon stream. Exemplary impurities include, but are not limited to heteroatoms (e.g., nitrogen and/or sulfur), metals (e.g., nickel and/or vanadium), asphaltenes, and combinations thereof.

In some embodiments, the dioxygenase may be capable of cleaving heteroatom-carbon bonds (e.g., nitrogen-carbon bonds, sulfur-carbon bonds) and/or carbon-carbon bonds, particularly, in non-porphyrin compounds, to release the impurities. It is contemplated herein that removal of impurities from the hydrocarbon stream also encompasses conversion of larger hydrocarbon compounds to smaller hydrocarbon compounds, which can also advantageously reduce viscosity of the hydrocarbon stream, as well as conversion of heteroatom containing compounds into compounds which can be more easily removed in further upgrading or refining processes, such as hydrotreating.

For example, with respect to asphaltenes, removal of asphaltenes may be accomplished by a dioxygenase described herein cleaving the multi-ring aromatics present in the asphaltenes, such that the asphaltenes are converted into smaller hydrocarbons thereby reducing asphaltene content (e.g., multi-ring aromatic content) in the hydrocarbon stream. For example, a dioxygenase described herein may be capable of converting larger nitrogen containing compounds into smaller nitrogen containing compounds, such as amines, which can be more easily removed in further upgrading or refining processes, such as hydrotreating. In some embodiments, methods of reducing content of multi-ring aromatic molecules in a hydrocarbon stream are provided herein comprising contacting the hydrocarbon stream with a dioxygenase and/or composition described herein.

In other embodiments, the upgrading methods described herein can enhance the quantity of hydrocarbons recovered from a hydrocarbon stream or limit the loss of hydrocarbons, for example, the dioxygenase described herein can selectively remove impurities from hydrocarbon compounds in the hydrocarbon stream without removing the entire hydrocarbon molecules, i.e., leaving the hydrocarbon backbone substantially untouched. Thus, in some embodiments, there can be lower loss of hydrocarbons following separation of the impurities from the hydrocarbon stream, for example, a loss of ≤15 wt %, ≤10 wt %, ≤8.0 wt %, ≤5.0 wt %, or ≤1.0 wt % of hydrocarbons may occur after separation of the impurities from the hydrocarbon stream.

Many of the enzymes described herein require a reducing agent (e.g., NADPH) co-factor to function. In certain embodiments, the enzymes make contact with the hydrocarbon stream in the presence of a reducing agent. In certain embodiments, the enzymes make contact with the hydrocarbon stream without the addition of reducing agents. Where a reducing agent is not added, the reducing power necessary for enzyme function can be supplied in some other manner, for example by passing a low power current through the environment while the enzymes are in contact with the hydrocarbon stream.

The hydrocarbon stream may be contacted with the dioxygenases and compositions described herein for any suitable amount of time. Advantageously, upgrading of the hydrocarbon stream when contacted with the dioxygenases described herein may occur in a short period of time, for example, the hydrocarbon stream may be contacted with dioxygenases for ≤about 10 hours, ≤about 5.0 hours, ≤about 1.0 hours, ≤about 30 minutes, ≤about 10 minutes, ≤about 1.0 minutes, ≤about 30 seconds, ≤about 10 seconds or ≤about 1.0 second.

Advantageously, the methods described here can be performed across a wide range of pressures and temperatures and even at ambient pressure and temperature. Effective upgrading conditions can include temperatures of about 15° C. to about 30° C. and pressures of from about 90 kPa to about 200 kPa. Additionally or alternatively, upgrading can be performed at higher temperatures of about 30° C. to about 200° C. or 30° C. to about 120° C.

### Locations, Forms and Immobilization

The methods described herein can be performed in various locations. For example, the dioxygenase may be present in an oil reservoir/wellbore, a pipeline, a tank, a vessel, a reactor, or any combinations thereof. In a particular embodiment, a dioxygenase may contact crude oil in the oil reservoir/wellbore, for example, through enzyme injection into the oil reservoir/wellbore. In another particular embodiment, the dioxygenase may contact a hydrocarbon stream, e.g., crude oil or hydrocarbon product stream, as it flows and/or resides in a pipeline and/or a holding vessel or a tank. When added to a pipeline and/or a holding vessel or a tank, a hydrocarbon stream may be upgraded without any substantially additional processing time, for example, when a hydrocarbon stream is awaiting further processing and/or transport.

In certain embodiments, the dioxygenases and compositions described herein can be present in free form or crystal form, while in other embodiments the dioxygenases and compositions can be immobilized on a carrier or scaffold, such as a membrane, a filter, a matrix, diatomaceous material, particles, beads, in an ionic liquid coating, an electrode, or a mesh.

In certain embodiments, the dioxygenases and compositions described herein can be present in crystal form and the crystals can be added to hydrocarbon streams at the various locations listed above. Standard techniques known to a person of ordinary skill in the art may be used to form dioxygenase crystals.

Additionally or alternatively, the dioxygenases and compositions described herein can be immobilized by standard techniques known to a person of ordinary skill in the art, and the hydrocarbon stream may contact an immobilized dioxygenase by flowing over, through, and/or around the immobilized dioxygenase. Suitable carriers or scaffolds include, but are not limited to a membrane, a filter, a matrix, diatomaceous material, particles, beads, an ionic liquid coating, an electrode, a mesh, and combinations thereof. In some embodiments, the matrix may comprise an ion-exchange resin, a polymeric resin and/or a water-wet protein attached to a hydrophilic surface, being a surface that is capable of forming an ionic or hydrogen bond with water and has a water contact angle of less than 90 degrees. For example, one or more dioxygenases may be present on a matrix with a thin layer of water-wet protein, which may maintain structure and function of the dioxygenase. In some embodiments, the particles and/or beads may comprise a material selected from the group consisting of glass, ceramic, and a polymer (e.g., polyvinyl alcohol beads). In some embodiments, one or more dioxygenases may be dispersed into heated and melted ionic liquids, and following cooling, the one or more dioxygenases may be coated in an ionic liquid, which may improve stability of a dioxygenase, for example, when contacted with organic solvents.

Additionally or alternatively, suitable carriers or scaffolds can comprise at least one transmembrane domain (e.g., alpha helical domain including hydrophobic residues, which can lock a dioxygenase within a matrix), at least one peripheral membrane domain (e.g., signal proteins), and combinations thereof along with the one or more dioxygenases. In other embodiments, the dioxygenase can be semi-immobilized in a packed bed of a reactor.

### Optional Method Steps

Additionally or alternatively, the methods can further comprise selecting one or more dioxygenases for contacting with the hydrocarbon stream based upon impurity type and content of the hydrocarbon stream. For example, the hydrocarbon stream may be tested to determine impurities content (e.g., nitrogen, sulfur, nickel and vanadium content) and properties. Then a dioxygenase or mixture of dioxygenases may be selected based on the impurities present in the hydrocarbon stream and properties of the hydrocarbon stream. The dioxygenase or mixture of dioxygenases may then be obtained or produced via methods known in the art, for example, the dioxygenase(s) may be produced in Escherichia coli, the cells may be used as whole cells or be lysed, and the soluble fraction may be removed.

In other embodiments, methods of enhanced oil recovery using one or more dioxygenase as described herein are provided. For example, one or more dioxygenase, singularly or in combination with an injection fluid, may be introduced to an oil reservoir/wellbore. In some embodiments, the one or more dioxygenase may reduce the viscosity of the oil present in the reservoir/wellbore allowing for increased oil recovery.

It is also contemplated herein that the dioxygenases described herein may be used in further refining processes, for example, the dioxygenases may be present in reactors for hydroprocessing, hydrofinishing, hydrotreating, hydrocracking, catalytic dewaxing (such as hydrodewaxing), solvent dewaxing, and combinations thereof.

### EXAMPLES

Without further description, it is believed that one of ordinary skill in the art can, using the preceding description and the following illustrative examples, make and utilize the enzymes and compositions described herein and practice the methods disclosed herein.

### Example 1: Strain Construction

To make the strains for tests described below, 7 mL of LB-Kanamycin (LB-Kan) per strain in 20 mL test tubes were innoculated with E. coli (B121) strains: untransformed E. coli; a strain transformed with pET28 empty vector; pET28-SEQ ID NO:2; pET28-SEQ ID NO:8; pET28-SEQ ID NO:14; pET28-SEQ ID NO:20; and pET28-SEQ ID NO:26. The inoculated samples were incubated for 16 hrs. at 37° C. with gentle shaking.

Duplicate samples of each inoculum were made by diluting 3 mL of each of these cultures in 200 mL LB-Kan in 500 mL sterile Erlenmeyer flasks. The flasks were incubated at 37° C. Once each culture reached OD₆₀₀=0.6, 40 μL of 1 M IPTG was added to induce protein expression. The flasks were then incubated overnight at room temperature with shaking.

The contents of each 500 mL flask were then transferred to 4×50 mL tubes. The tubes were centrifuged at 3000×g for 30 min. The media were decanted off. The pellets were resuspended in 5 mL each of M9 solution with vortexing. These samples were then centrifuged again at 3000×g for 15 min. The M9 media were decanted and replaced with a fresh 4 mL of M9 media per tube. The pellets were again resuspended with vortexin, and all samples of each strain pooled into a single 50 mL tube per strain.

The optical density (OD₆₀₀) of each cell suspension at 50×, 100×, and 200× dilutions (1 mL per measurement). The cell pellets were lysed by sonication at amplitude=100% (5 cycles of 15 sec. pulse, followed by 30 sec. rest).

### Example 2: Assay of Enzyme Activity on Heterocyclic Organic Compounds

To test the ideas discussed above, lysates from each strain were inoculated into four different fuel compositions containing undesirable impurities: carbazole; dibenzothiophene; dibenzofuran; and fluorene. 4 mL of each lysed cell suspension was transferred to a 25 mL flask, and M9 medium was added to bring the volume to 4.95 mL. 50 μL of 1% stock solution of: carbazole; dibenzothiophene (DBT); 4-methyl DBT; 4,6-dimethyl DBT; dibenzofuran; 9-fluorene; or 3-ethyl carbazole was added to each. Each flask was incubated at 30° C. with shaking at 60 RPM.

Thirty five microliters of 6 N HCl and 20 mL of ethyl acetate was added to each flask, and the tubes swirled gently. One and a half milliliters of the ethyl acetate layer (i.e., the top layer) was removed to a 3 mL syringe with nylon filter. The ethyl acetate was filtered into a labeled HPLC vial for analysis. HPLC was analyzed using the 50-100% methanol gradient method.

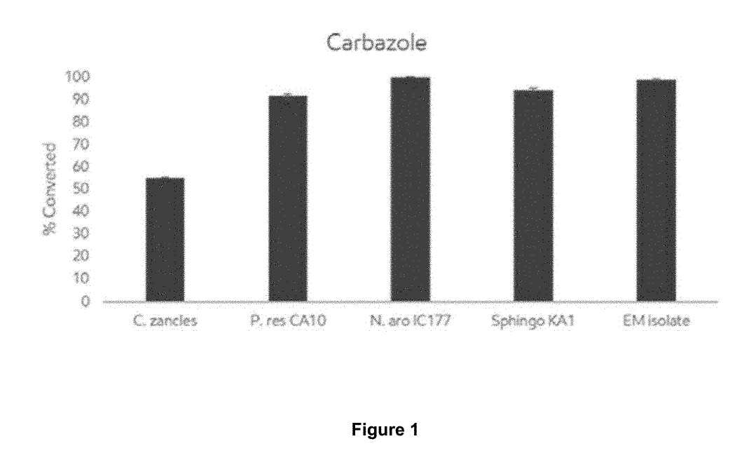

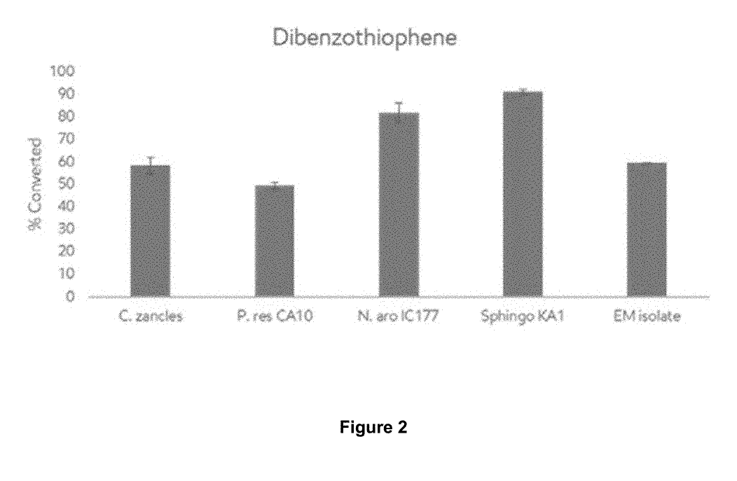

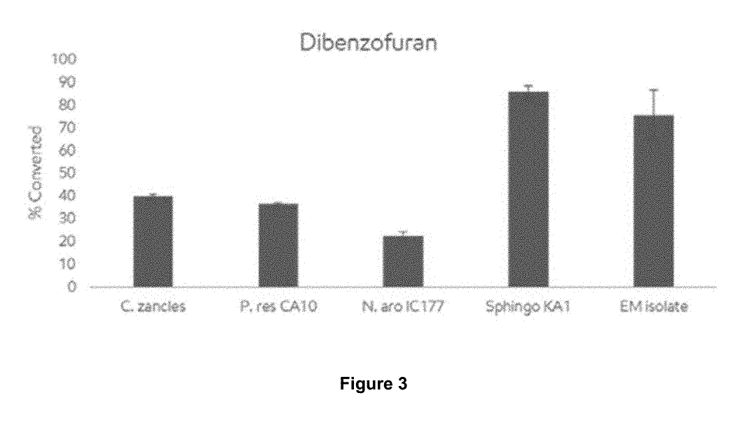

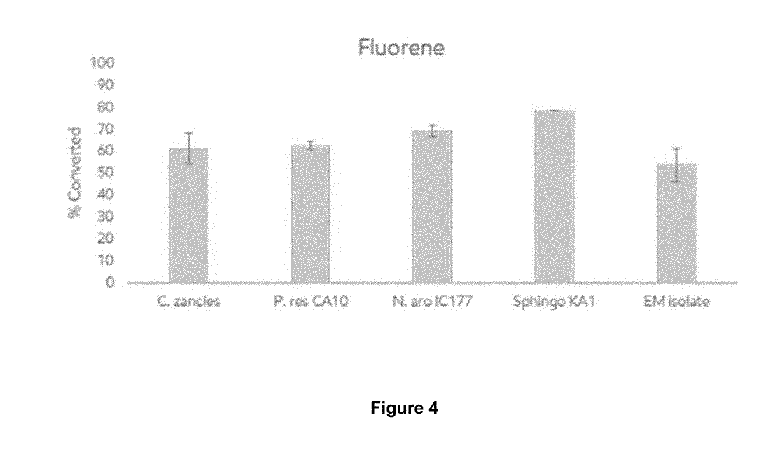

As shown in FIGS. 1-4, different enzyme constructs had greater or lesser effects on each impurity. Additional results are shown in Table 1 below.

TABLE 1
Effect of enzymes on polycyclic aromatic hydrocarbon (% conversion)

| | | | | 4,6- | | | |
|---|---|---|---|---|---|---|---|
| | | | 4-Methyl | Dimethyl | | 9- | 3-Ethyl |
| Strain | Carbazole | DBT | DBT | DBT | Dibenzofuran | Fluorene | carbazole |
| Untransformed | 13.174 | 49.415 | 67.008 | 84.276 | 32.646 | 35.206 | 62.612 |
| Empty vector | 20.353 | 50.236 | 67.564 | 85.284 | 26.496 | 39.314 | 60.087 |
| SEQ ID NO: 2 | 85.697 | 55.526 | 65.14 | 82.749 | 49.297 | 49.432 | 86.023 |
| SEQ ID NO: 8 | 91.624 | 75.976 | 70.658 | 83.699 | 89.179 | 100 | 86.79 |
| SEQ ID NO: 14 | 98.859 | 74.325 | 78.592 | 83.278 | 82.005 | 98.435 | 98.154 |
| SEQ ID NO: 20 | 88.075 | 70.122 | 79.31 | 69.221 | 71.346 | 89.815 | 84.627 |

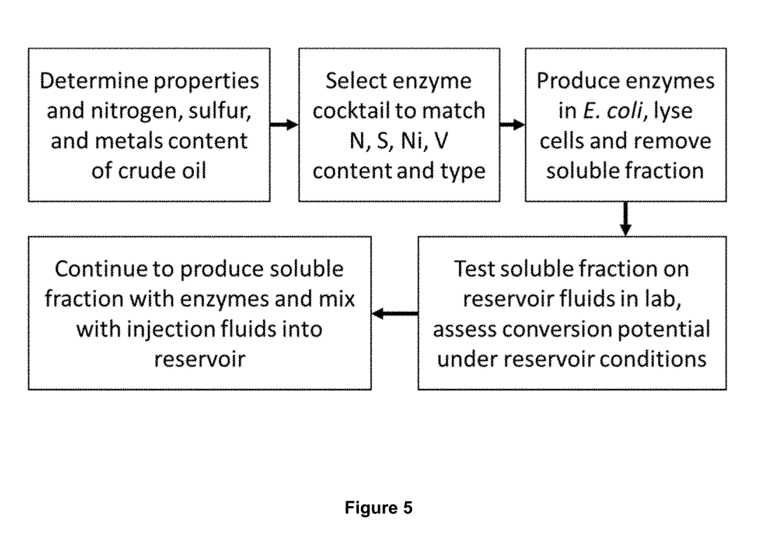

These results confirm that a variety of biologically derived enzymes are available to remediate impurities in less refined fuel sources. Based on screens of the sort exemplified in FIGS. 1-4, it is possible to produce enzymes and process hydrocarbom streams according to the methods disclosed herein (see, e.g., FIG. 5).

TABLE 2
SEQ ID NO correspondences

| SEQ ID NO | | | |
|---|---|---|---|
| Nucleotide | Protein | Gene | Organism |
| 1 | 2 | CarAa | Pseudomonas resinovorans |
| 3 | 4 | CarAc | |
| 5 | 6 | CarAd | |
| 7 | 8 | CarAa | Sphingomonas sp. KA1 |
| 9 | 10 | CarAc | |
| 11 | 12 | CarAd | |
| 13 | 14 | CarAa | Sphingosinicella sp. JP1 |
| 15 | 16 | CarAc | |
| 17 | 18 | CarAd | |
| 19 | 20 | CarAa | Nocardiodes aromaticivorans |
| 21 | 22 | CarAc | |
| 23 | 24 | CarAd | |
| 25 | 26 | CarAa | Cycloclasticus zancles |
| 27 | 28 | CarAc | |
| 29 | 30 | CarAd | |
| 31 | 32 | CarAa | Pseudoxanthomonas spadix |
| 33 | 34 | CarAc | |
| 35 | 36 | CarAd | |
| 37 | 38 | CarAa | Paraburkholderia xenovorans |
| 39 | 40 | CarAa | Sphingomonas sp. CB3 |
| 41 | 42 | CarAa | Terrabacter sp. YK3 |
| 43 | 44 | CarAa | Unknown soil isolate |
| 45 | 46 | CarAa | Rhodococcus opacus |
| 47 | 48 | CarAa | Nocardiodes sp. KP7 |

1. A method of biologically upgrading a hydrocarbon stream comprising contacting the hydrocarbon stream with an EC1.14.12 dioxygenase.

2. The method of claim 1, wherein the dioxygenase is substantially cell-free.

3. The method of claim 1, wherein the dioxygenase is a recombinant enzyme.

4. The method of claim 1, wherein the dioxygenase classifies as belonging to subfamily cd08881.

5. The method of claim 4, wherein the dioxygenase classifies as belonging to Pfam family PFAM00848 or PFAM11723.

6. The method of claim 1, wherein the dioxygenase is capable of cleaving heteroatom-carbon bonds and carbon-carbon bonds in non-porphyrin compounds.

7. The method of claim 1, wherein the dioxygenase has at least 85% sequence identity to a dioxygenase selected from the group consisting of SEQ ID NOs: 2, 8, 14, 20, 26, 32, 38, 40, 42, 44, 46, and 48.

8. The method of claim 7, further comprising contacting the hydrocarbon stream with an enzyme having at least 85% sequence identity to a polypeptide selected from the group consisting of SEQ ID NOs: 4, 10, 16, 22, 28, and 34.

9. The method of claim 8, further comprising contacting the hydrocarbon stream with an enzyme having at least 85% sequence identity to a polypeptide selected from the group consisting of SEQ ID NOs: 6, 12, 18, 24, 30, and 36.

10. The method of claim 1, wherein the biological upgrading comprises removing impurities from the hydrocarbon stream.

11. The method of claim 10, wherein the impurities comprise metal, heteroatoms, asphaltenes, or a combination thereof.

12. The method of claim 11, wherein the metal is nickel or vanadium.

13. The method of claim 11, wherein the heteroatom is nitrogen or sulfur.

14. The method of claim 1, wherein the hydrocarbon stream is crude oil or vacuum resid.

15. The method of any one of the previous claims, wherein the contacting is performed at a temperature from about 15° C. to about 90° C.

16. The method of claim 1, wherein the dioxygenase is thermally stable from about 90° C. to about 120° C.

17. The method of claim 1, further comprising selecting one or more dioxygenases for the contacting step based upon impurity type and content of the hydrocarbon stream.

18. The method of claim 1, wherein there is less than 10 wt % loss of hydrocarbon following separating the impurities from the hydrocarbon stream.

19. The method of claim 1, wherein the dioxygenase is present in an oil reservoir, a pipeline, a tank, a vessel, and/or a reactor.

20. The method of claim 1, wherein the dioxygenase is in free form, crystal form, and/or immobilized on a carrier.

21. The method of claim 20, wherein the carrier is selected from the group consisting of a membrane, a filter, a matrix, diatomaceous material, particles, beads, an ionic liquid, an electrode, a mesh, and a combination thereof.

22. The method of claim 21, wherein the matrix comprises an ion-exchange resin, a polymeric resin and/or a water wet protein.

23. The method of claim 21, wherein the particles and/or beads comprise a material selected from the group consisting of glass, ceramic, and a polymer.

24. The method of claim 1, wherein the dioxygenase is hydrophobically modified to be at least 10% more enriched in hydrophobic amino acids selected from the group consisting of Ala, Gly, Ile, Leu, Met, Pro, Phe, and Trp.

25. The method of claim 24, wherein the dioxygenase is selected from the group consisting of SEQ ID NOs: 2, 8, 14, 20, 26, 32, 38, 40, 42, 44, 46, and 48.

26. The method of claim 24, wherein the enrichment is at least 20%.

27. The method of claim 24, wherein enrichment is achieved by replacing a native residue with the hydrophobic amino acid.

28. The method of claim 24, wherein enrichment is achieved by adding the hydrophobic amino acid between two native residues.

29. The method of claim 1, wherein the dioxygenase is rinsed with n-propanol.

30. The method of claim 1, wherein the dioxygenase is conjugated to a polyethylene glycol.

31. The method of claim 1, wherein disulfide bridges are added to the dioxygenase.

32. The method of claim 1, wherein one to ten hydrophobic amino acid residues are added to an amino or carboxy terminus of the dioxygenase, wherein the hydrophobic amino acid is selected from the group consisting of Ala, Gly, Ile, Leu, Met, Pro, Phe, and Trp.

33. A recombinant polypeptide having at least 70% sequence identity but no more than 90% sequence identity to any one of SEQ ID NOs: 2, 8, 14, 20, 26, 32, 38, 40, 42, 44, 46, or 48, wherein the sequence is manipulated to be at least 10% more enriched in hydrophobic amino acids relative to the sequence selected from SEQ ID NOs: 2, 8, 14, 20, 26, 32, 38, 40, 42, 44, 46, and 48, and wherein the hydrophobic amino acids are selected from the group consisting of Ala, Gly, Ile, Leu, Met, Pro, Phe, and Trp.

34. The recombinant polypeptide of claim 33, wherein the enrichment is at least 20%.

35. A polypeptide having at least 70% sequence identity to any one of SEQ ID NOs: 14, 16, or 18.

36. An isolated or recombinant nucleic acid molecule comprising a sequence encoding the polypeptide of claim 33.

37. A vector comprising the nucleic acid molecule of claim 36.
